(19) United States
(12) Patent Application Publication
(10) Pub. No.: US 2013/0102580 A1
(43) Pub. Date: Apr. 25, 2013

(54) COMPOSITIONS AND METHODS RELATED TO DEOXYCHOLIC ACID AND ITS POLYMORPHS

(71) Applicant: Kythera Biopharmaceuticals, Inc., Calabasas, CA (US)

(72) Inventors: Achampeta Rathan Prasad, Calabasas, CA (US); Sankar Subramanian, Calabasas, CA (US); Nicholas Holman, Calabasas, CA (US); John Gregory Reid, Groton, MA (US); Steven Pfeiffer, Calabasas, CA (US); Xufeng Sun, Calabasas, CA (US); John Knight, Calabasas, CA (US); Randy Steinbrink, Calabasas, CA (US)

(73) Assignee: Kythera Biopharmaceuticals, Inc., Calabasas, CA (US)

(21) Appl. No.: 13/623,772

(22) Filed: Sep. 20, 2012

Related U.S. Application Data
(60) Provisional application No. 61/538,084, filed on Sep. 22, 2011.
(60) Provisional application No. 61/558,375, filed on Nov. 10, 2011.
(60) Provisional application No. 61/659,920, filed on Jun. 14, 2012.

(51) Int. Cl. C07J 9/00

(52) U.S. Cl. CPC C07J 9/005
USPC 514/182; 552/553

(57) ABSTRACT

Provided herein are polymorphic forms of deoxycholic acid (DCA), improved methods of synthesizing DCA and intermediates thereto, and compositions and fat removal methods employing the DCA as provided herein.

### CROSS REFERENCE TO RELATED APPLICATIONS

This application claims the benefit under 35 U.S.C. 119(e) of U.S. Provisional Application Ser. Nos. 61/538,084, filed Sep. 22, 2011; 61/558,375, filed Nov. 10, 2011; and 61/659,920, filed Jun. 14, 2012, each of which is hereby incorporated by reference into this application in its entirety.

### FIELD OF THE INVENTION

Provided herein are polymorphic forms of deoxycholic acid (DCA), improved methods of synthesizing DCA and intermediates thereto, and compositions and fat removal methods employing the DCA as provided herein. Thus, in certain aspects, this invention provides DCA polymorphs, preferably, surprisingly water and thermostable crystalline anhydrate polymorphs of DCA. In other aspects, this invention further provides purified DCA compositions, and processes and compositions useful for DCA purification wherein the DCA has a purity, preferably, of at least 99%. In yet other aspects, this invention provides compounds, compositions, and processes related to preparation of synthetic DCA.

### STATE OF THE ART

Rapid removal of body fat is an age-old ideal, and many substances have been claimed to accomplish such results, although few have shown results. “Mesotherapy”, or the use of injectables for the removal of fat, is not widely accepted among medical practitioners due to safety and efficacy concerns, although homeopathic and cosmetic claims have been made since the 1950's. Mesotherapy was originally conceived in Europe as a method of utilizing cutaneous injections containing a mixture of compounds for the treatment of local medical and cosmetic conditions. Although mesotherapy was traditionally employed for pain relief, its cosmetic applications, particularly fat and cellulite removal, have recently received attention in the United States. One such reported treatment for localized fat reduction, which was popularized in Brazil and uses injections of phosphatidylcholine, has been erroneously considered synonymous with mesotherapy. Despite its attraction as a purported “fat-dissolving” injection, there is little safety and efficacy data of these cosmetic treatments. See, Rotunda, A. M. and M. Kolodney, Dermatologic Surgery 32: 465-480 (2006) (“Mesotherapy and Phosphatidylcholine Injections Historical Clarification and Review”).

Recently published literature reports that the bile acid, DCA, and salts thereof, have fat removing properties when injected into fatty deposits in vivo. See, WO 2005/117900 and WO 2005/112942, as well as US2005/0261258; US2005/0267080; US2006/127468; and US20060154906, each of which is incorporated herein by reference in its entirety). Deoxycholate injected into fat tissue degrades fat cells via a cytolytic mechanism. Because deoxycholate injected into fat is rapidly inactivated by exposure to protein and then rapidly returns to the intestinal contents, its effects are spatially contained. As a result of this attenuation effect that confers clinical safety, fat removal typically require 4-6 sessions. This localized fat removal without the need for surgery is beneficial not only for therapeutic treatment relating to pathological localized fat deposits (e.g., dyslipidemias incident to medical intervention in the treatment of HIV), but also for cosmetic fat removal without the attendant risk inherent in surgery (e.g., liposuction). See, Rotunda et al., Dermatol. Surgery 30: 1001-1008 (2004) (“Detergent effects of sodium deoxycholate are a major feature of an injectable phosphatidylcholine formulation used for localized fat dissolution”) and Rotunda et al., J. Am. Acad. Dermatol. (2005: 973-978) (“Lipomas treated with subcutaneous deoxycholate injections”), both incorporated herein by reference in their entirety. U.S. Pat. Nos. 7,622,130 and 7,754,230 describe using DCA for fat removal.

In addition, many important steroids have a 12-α-hydroxy-substituent on the C-ring of the steroid. Such compounds include, by way of example, bile acids such as DCA, cholic acid, lithocholic acid, and the like. Heretofore, such compounds were typically recovered from bovine and ovine sources which provided a ready source of bile acids on a cost effective basis. However, with the recent discovery that pathogens such as prions can contaminate such sources, alternative methods for the synthesis of bile acids from plant sources or synthetic starting materials have become increasingly important. For example, DCA from animals in New Zealand are a source of bile acids for human use under US regulatory regimes, as long as the animals continue to remain isolated and otherwise free of observable pathogens. Such stringent conditions impose a limitation on the amount of suitable mammalian sourced bile acids and does not preclude the possibility that the bile acid will be free of such pathogens. U.S. Pat. No. 8,242,294 relates to DCA containing less than 1 ppt ¹⁴C.

There remains a need for suitable quantities of bile acids such as DCA, preferably for human administration Accordingly, there is an ongoing need to provide processes for preparing and purifying DCA.

Furthermore, when used for human administration, it is important that a crystalline agent like DCA retains its polymorphic and chemical stability, solubility, and other physicochemical properties over time and among various manufactured batches of the DCA. If the physicochemical properties vary with time and among batches, the administration of an effective dose becomes problematic and may lead to toxic side effects or to ineffective administration. Therefore, it is important to choose a form of the crystalline agent that is stable, is manufactured reproducibly, and has physicochemical properties favorable for its use for human administration. For a compound such as DCA, its solvated polymorphs may contain an organic solvent in an amount that is undesirable for human administration. However, removing such residual solvents from DCA crystals may be problematic. Accordingly, the use of such solvents for crystallizing DCA, particularly for preparing the drug substance or active pharmaceutical ingredient (API) are unpredictable and are limited.

Furthermore, the art remains unable to predict which crystalline form of an agent in general, and of DCA in particular, will have a combination of the desired properties and will be suitable for human administration, and how to make the agent in such a crystalline form.

### SUMMARY OF THE INVENTION

Provided herein are polymorphic forms of deoxycholic acid (DCA), improved methods of synthesizing DCA and intermediates thereto, and compositions and fat removal methods employing such DCA as provided herein.

Thus, in one aspect, this invention provides DCA polymorphs, preferably, surprisingly water-stable and thermostable crystalline anhydrate polymorphs of DCA.

Provided herein are crystalline polymorphs of DCA such as polymorphs of Forms A, B, C, and D, as characterized herein. Upon heating, the following polymorphic form conversions were observed: C→B→D→A, indicating that Form A was the most thermodynamically stable polymorph. And yet, surprisingly, when Forms A and B were slurried in about 1:1.2 v/v Ethanol (EtOH)/water at ambient temperature, Form A converted to Form C but Form B did not.

Based on a 2.4% water loss observed between 40 and 160° C. in its thermogravimetric analysis (TGA), Form C is contemplated to contain half a mole of loosely bound water per mole of DCA. Since none of Forms A, B, and D demonstrated any substantial water loss in their TGA, and since the hemihydrate form C is converted to Form B upon heating, and Form B is further converted to Forms D and A upon heating, Forms A, B, and D are anhydrous polymorphic forms. Based on its differential scanning calorimetry (DSC), Form A appears to be an ansolvate because it demonstrates a single endothermic peak in the DSC (see FIG. 6).

In one embodiment, the crystalline anhydrate DCA polymorph provided herein is of Form A. In another embodiment, the Form A polymorph is characterized by a powder X-ray diffraction peak at 15.0° 2theta, or by 1, 2 or 3 PXRD peaks selected from 8.9, 10.7, 14.0, 15.0, 16.2, and 19.1° 2theta. In another embodiment, the Form A polymorph is characterized by a PXRD pattern substantially as shown in FIG. 1. In another embodiment, the Form A is characterized by an endothermic peak (within ±2° C.) at 174° C. as measured by differential scanning calorimetry. In another embodiment, the Form A is characterized by the substantial absence of thermal events at temperatures below the endothermic peak at (174±2)° C., or above the endothermic peak up to a temperature of 300° C. as measured by differential scanning calorimetry.

In another embodiment, the crystalline anhydrate DCA polymorph provided herein is of Form B. In one embodiment, the Form B polymorph is characterized by a powder X-ray diffraction (PXRD) peak at 7.4° 2theta, or by 1, 2, or 3 PXRD peaks selected from 6.7, 7.3, 7.4, 8.4, 9.3, 11.2, 12.9, 13.9, 14.4, 14.6, 14.8, 15.8, 16.0, 16.9, and 17.8° 2theta. In another embodiment, the Form B polymorph is characterized by a PXRD pattern substantially as shown in FIG. 2. In another embodiment, the Form B is characterized by an endothermic peak (within ±2° C.) at 135° C. as measured by differential scanning calorimetry.

In another aspect, this invention provides a crystalline hydrate polymorph C of DCA. In another embodiment, the Form C polymorph is characterized by a powder X-ray diffraction peak at 15.8° 2theta, or by 1, 2, or 3 PXRD peaks selected from 6.6, 7.3, 7.4, 9.6, 9.9, 12.6, 13.0, 13.2, 13.9, 14.2, 15.1, 15.6, 15.8, 16.4, 17.0, 17.1, and 17.6° 2theta. In another embodiment, the Form C polymorph is characterized by a PXRD pattern substantially as shown in FIG. 3. In another embodiment, the Form C is characterized by a broad transition at under 100° C. as measured by differential scanning calorimetry. In another embodiment, the Form C polymorph is characterized by a transition corresponding to about 2.4% mass loss at a temperature of 40-140° C. in a TGA analysis.

In another embodiment, the crystalline anhydrate DCA polymorph provided herein is of Form D. In another embodiment, the Form D polymorph is characterized by a powder X-ray diffraction (PXRD) peak at 10.0° 2theta, or by 1, 2, or 3 PXRD peaks selected from 7.0, 7.4, 10.0, 14.2, 15.3, 15.8, 16.6, and 17.3° 2theta. In another embodiment, the Form D polymorph is characterized by a PXRD pattern substantially as shown in FIG. 5. In another embodiment, the Form D is characterized by an endothermic peak (within ±2° C.) at 156° C. as measured by differential scanning calorimetry.

In another aspect, this invention provides a DCA polymorph, preferably a crystalline anhydrate polymorph of DCA admixed with at least a pharmaceutically acceptable excipient. In one embodiment, the DCA polymorph is of Form B. In another embodiment, the DCA polymorph is Form A or D. In another embodiment, the polymorph admixed substantially excludes a hydrate polymorph, preferably, the polymorphic Form C. In another embodiment, the admixed composition comprises about 0.1% w/v to about 2% w/v, or preferably about 0.5% w/v to about 1.5% w/v DCA. In another embodiment, the admixed composition is an aqueous formulation suitable for subcutaneous injection. In another embodiment, the at least one pharmaceutically acceptable excipient and/or carrier is selected from the group consisting of water, a buffer, and a preservative.

In another aspect, provided herein are methods of converting one polymorphic form of DCA to another. In one embodiment, the Form C polymorph is heated under vacuum (e.g., about 50 mm of Hg) at a temperature under 135° C., preferably under 100° C., more preferably at about 40° C. to provide the Form B polymorph.

Within the various composition, method, and process aspects and embodiments provided herein, in one embodiment, the DCA utilized herein is non-microbial and/or non-mammalian DCA. Such DCA, which is synthetic in nature, in one embodiment, includes a sidechain:

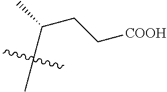 [SMILES: *[C@H](C)CCC(=O)O]

or an ester thereof that is incorporated synthetically into the DCA molecule. In another embodiment, such synthetic DCA is DCA that is not admixed with any cholic acid. As used herein, “non-microbial” refers to DCA that is not prepared microbially. In a preferred embodiment, the “non-microbial” DCA is not prepared using cholic acid. As used herein, “non-mammalian” refers to DCA that is not isolated from mammalian sources, non-limiting examples of which mammals include sheep and cattle. In another embodiment, the non-microbial and/or non-mammalian DCA utilized herein contain less than 1 ppt, preferably less than 0.9 ppt ¹⁴C.

In other aspects, this invention further provides purified DCA compositions, and processes and compositions useful for DCA purification wherein the DCA has a purity, preferably, of at least 99%. Various solvent systems were evaluated for crystallization and purification of DCA. While DCM/MeOH was suitable for providing purified DCA, removing dichloromethane (DCM) from DCA crystallized from DCM/MeOH was problematic; therefore DCA purified initially from DCM/MeOH was preferably recrystallized to obtain a crystal form with low residual organic solvents.

To this end, DMSO crystallization showed high levels of residual DMSO. Acetone crystallization showed poor recovery of DCA. EtOH/water, methyl ethyl ketone (MEK)/n-heptane and isopryl alcohol (IPA)/n-heptane were also tested as crystallization solvents. The MEK/n-heptane system provided purification and recovery but residual MEK could not be removed. The IPA/n-heptane system provided purification, recovery, and volume efficiency but residual IPA could not be removed. In view of the failures of the other solvent systems, surprisingly, the EtOH/water system provided good purification, volume efficiency, and recovery with no residual solvent issue for crude DCA containing up to 0.54% of DS-DCA.

In yet other aspects, this invention provides compounds, compositions, and processes related to preparation of synthetic DCA. In such aspects, provided herein are compounds, compositions, and processes related to preparation of synthetic DCA. One of the advantages of these processes, compositions, and intermediates is that, they involve an internal 3,9steroidal ketal, which is obtained easily according to this invention and undergoes olefination at a 17-position keto group without requiring additional functional group protections. Another of the advantages of the processes provided herein is that the improved allylic oxidation of 128 under various conditions provide 129. Under certain conditions, a two-step process, where an under oxidized allylic alcohol 128a was oxidized to 129, was found to be preferable to a one-step process. Also provided herein are pharmaceutical compositions for and methods of removing fat deposit employing the compositions and polymorphs of this invention.

In one of its compound aspects, this invention provides a compound selected from the group consisting of:

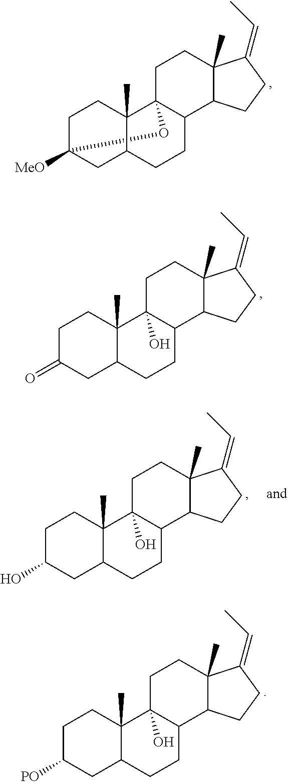

In another of its compound aspects, this invention provides a compound of formula DS-DCA:

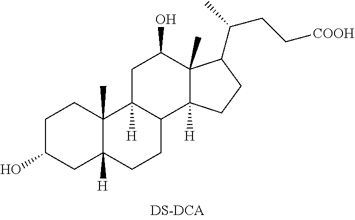

or a C₁-C₆ alkyl ester or a salt thereof, which salt includes, but is not limited to, a pharmaceutically acceptable salt. In one embodiment, this invention provides the DS-DCA, the C₁-C₆ alkyl ester or the salt thereof, admixed with DCA or a C₁-C₆ alkyl ester or a salt thereof. In one embodiment, the DS-DCA is non-microbial and/or non-mammalian DS-DCA. In another embodiment, the DS-DCA has a ¹⁴C level less than 1 ppt. In another embodiment, this invention provides DCA that contain less than 0.5% w/w, preferably less than 0.1% w/w, more preferably less than 0.05% w/w of DS-DCA.

In one of its composition aspects, this invention provides a composition comprising a compound of formula:

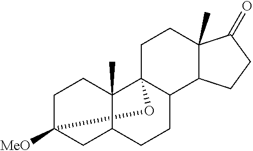

and a 2 carbon olefination reagent.

In another of its composition aspects, this invention provides a composition comprising a compound of formula:

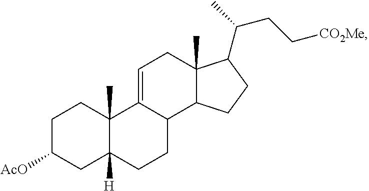 [SMILES: COC(=O)CC[C@@H](C)C1CCC2C3CC[C@@H]4C[C@H](OC(C)=O)CC[C@]4(C)C3=CC[C@@]21C]

tertiarybutyl hydroperoxide, and CuI. In one embodiment, the composition is free of hypochlorite (OCl(−)).

In another of its composition aspects, this invention provides a composition comprising a compound of formula:

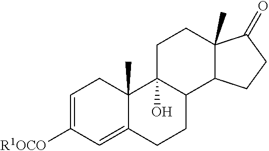

wherein R¹ is C₁-C₆ alkyl optionally substituted with 1-3 halo, preferably fluoro, and/or alkoxy groups, or is aryl, optionally substituted with 1-3 C₁-C₃ alkyl, halo, preferably fluoro, and/or alkoxy groups, and a hydrogenation catalyst: preferably palladium, platinum, or such other metal, or an oxide or hydroxide of each thereof, supported on carbon, alumina, or such other support. In some embodiments, the composition further comprises hydrogen. In some embodiments, the composition further comprises a solvent, preferably, any inert solvent that does not react with hydrogen in the presence of a hydrogenation catalyst, such as dimethyl formamide, dimethyl acetamide, C₁-C₄ alcohols, ethyl acetate, tetrahydrofuran, and the like.

In another of its composition aspects, this invention is directed to compositions comprising DCA or a salt thereof and a mixture of one or more C₁₋₃ alcohol(s) and deionized water. In a preferred embodiment the C₁₋₃ alcohol is ethanol. In a more preferred embodiment, the ethanol and the water is present in ratio of about 1:1 to about 5:1 v/v.

In one of its process aspects, this invention provides a process of oxidizing a 12-position methylene group of a steroid which methylene group is adjacent to a Δ-9,11-ene, the method comprising contacting the steroid containing the methylene group with tertiarybutyl hydroperoxide and CuI under conditions to provide a 12-hydroxy Δ-9,11-ene steroid and optionally a 12-keto Δ-9,11-ene steroid. In one embodiment, the method further comprises contacting the 12-hydroxy Δ-9,11-ene steroid with pyridinium chlorochromate under conditions to provide the 12-keto Δ-9,11-ene steroid.

In another of its process aspects, this invention provides a process of preparing DCA:

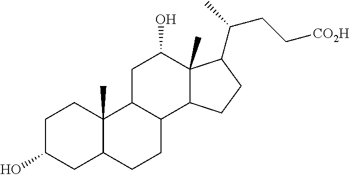 [SMILES: C[C@H](CCC(=O)O)C1CCC2C3CCC4C[C@H](O)CC[C@]4(C)C3C[C@H](O)[C@@]21C]

or a salt thereof, the process comprising,
(i) contacting a compound of formula 121:

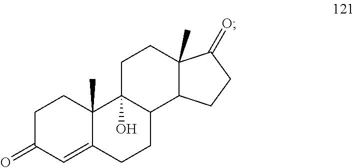

with H₂ under hydrogenation condition in a solvent comprising MeOH to form a compound of formula 121a:

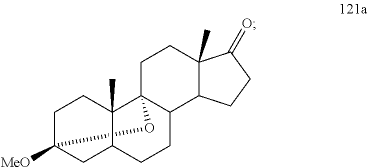

(ii) contacting the compound of formula 121a with a 2 carbon olefination ragent under olefin forming condition to provide a compound of formula 121b:

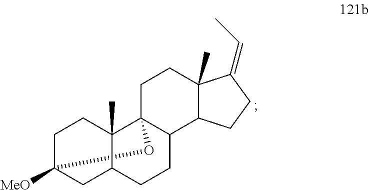 [SMILES: C/C=C1/CCC2C3CCC4C[C@]5(OC)CC[C@]4(C)[C@]3(CC[C@]12C)O5]

(iii) contacting a compound of formula 121b with an aqueous acid under ketal hydrolysis conditions to provide a compound of formula 121c:

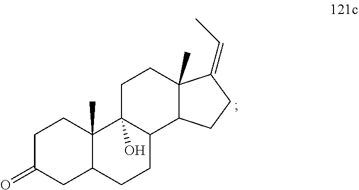

(iv) contacting the compound of formula 121c with a reducing agent to provide a compound of formula 121e:

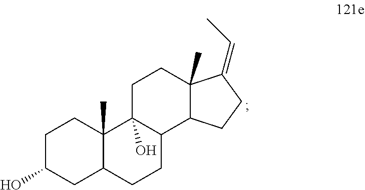

(v) converting the compound of formula 121e to a compound of formula 121f, wherein P is a hydroxy protecting group:

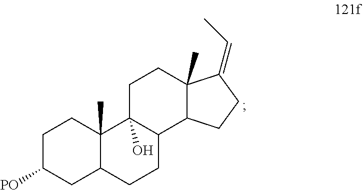

(vi) contacting the compound 121f under dehydrating conditions to provide a compound of formula 126:

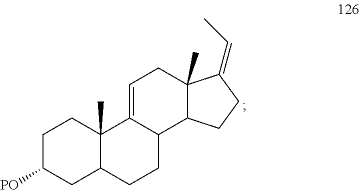

(vii) contacting the compound 126 with an alkyl propiolate of formula HCCCO₂R or an alkyl acrylate of formula H₂CCHCO₂R in presence of a Lewis acid catalyst to provide a compound of formula 127a, wherein R is alkyl optionally substituted with 1-3 aryl groups and [symbol] refers to a single (as obtained from the acrylate) or a double (as obtained from the propiolate) bond:

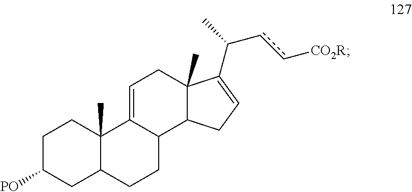

(viii) contacting the compound of formula 127 with H₂ under hydrogenation conditions to form a compound of formula 128:

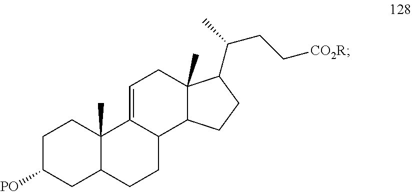

(ix) contacting the compound of formula 128 with an oxidizing agent under allylic oxidation conditions to provide a compound of formula 128a, or 129, or a mixture of compounds 128a and 129:

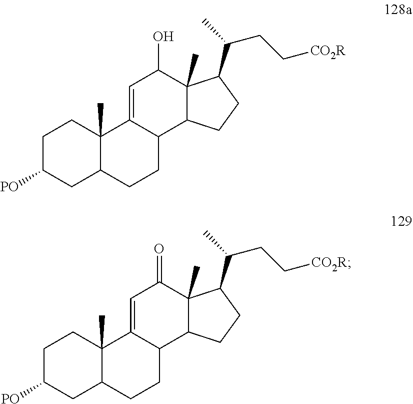

(x) optionally, preferably when the compound of formula 128a is present in a substantial amount in the mixture, contacting the mixture with an oxidizing agent under oxidizing conditions to provide the compound of formula 129;
(xi) contacting the compound of formula 129 with hydrogen under hydrogenation condition to provide a compound of formula 130 optionally admixed with a compound of formula 130a:

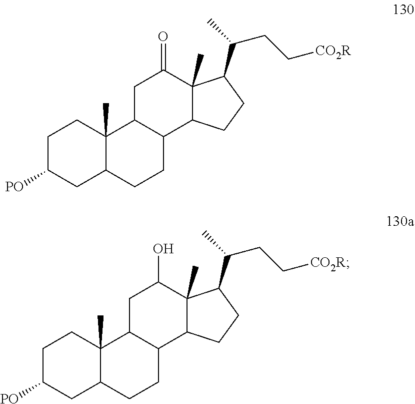

(xii) optionally, preferably when the compound of formula 130a is admixed in a substantial amount, contacting the compound of formula 130 admixed with the compound of formula 130a with an oxidizing agent under oxidizing conditions to provide the compound of formula 130;
(xiii) contacting the compound of formula 130 with a reducing agent to provide a compound of formula 131:

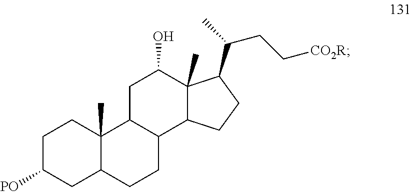

(xiv) deprotecting the protected alcohol and the carboxylic acid ester groups of the compound of formula 131 under deprotecting conditions to provide DCA or a salt thereof.

In one embodiment, the solvent comprising MeOH is MeOH. In another embodiment, the 2 carbon olefination reagent comprises EtPPh₃Br and tertiarybutoxide. In another embodiment, the reducing agent in step (iv) is a borohydride, preferably, NaBH₄. In another embodiment, P is R²—CO—, wherein R² is C₁-C₆ alkyl or aryl, wherein the alkyl and the aryl are optionally substituted with 1-3 aryl, C₁-C₆ alkoxy, and/or halo. In another embodiment, the Lewis acid catalyst is EtAlCl₂. In another embodiment, the dehydration condition comprises contacting with an acid or with thionyl chloride. In another embodiment, the hydrogenation condition comprises employing a supported Pd, Pt, or Rh catalyst. In another embodiment, the oxidation in step (ix) is performed using a hydroperoxide and a Cu(I) salt. In another embodiment, the oxidation in step (x) is performed using pyridinium chlorochromate (PCC), preferably under anhydrous conditions. In another embodiment, the optional oxidation in step (xii) is performed with PCC. In another embodiment, the reducing in step (xiii) is performed with LiAl(OCMe₃)₃H. In another embodiment, the deprotection is performed with aqueous alkali.

In certain other of its process aspects, this invention provides methods related to stereoselectively reducing a steroid containing 3-keto group and a 4,5-ene unsaturation to provide a 3-alpha-hydroxy and 5-beta-H steroid or a 3-ester thereof. In one such aspect, this invention provides a method of synthesis comprising contacting a compound of formula:

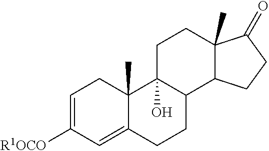

with a hydrogenation catalyst and hydrogen under conditions to provide a compound of formula:

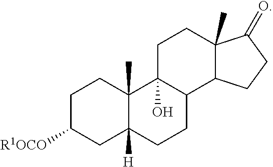

It is contemplated that the 9-hydroxy and the 17-keto groups present in the compounds utilized in this invention can be suitably protected or derivatized. For example, the hydroxy group can be protected to form an ester (—OCOR¹) or a silyl ether (—OSi(R¹)₃) wherein each R′ is independently C₁-C₆ alkyl optionally substituted with 1-3 halo, preferably fluoro, and/or alkoxy groups, or is aryl, optionally substituted with 1-3 C₁-C₃ alkyl, halo, preferably fluoro, and/or alkoxy groups.

In one of its fat removal method aspects, this invention provides a method for reducing a subcutaneous fat deposit in a subject comprising administering locally to the fat deposit in the subject, under a condition to dissolve the fat deposit, an effective amount of a crystalline anhydrate form, preferably Form B DCA, admixed with at least a pharmaceutically acceptable excipient. As used herein, Pharmaceutically acceptable excipient includes pharmaceutically acceptable alkali, such as sodium or potassium hydroxide.

These and other aspects and embodiments of this invention are disclosed hereinbelow.

### BRIEF DESCRIPTION OF THE FIGURES

FIG. 1 illustrates a PXRD pattern of Form A polymorph of DCA.

FIG. 2 illustrates a PXRD pattern of Form B polymorph of DCA.

FIG. 3 illustrates a PXRD pattern of Form C polymorph of DCA.

FIG. 4 illustrates a PXRD stack plot of thermal conversion of Form C to Form B DCA.

FIG. 5 illustrates a PXRD pattern of Form D polymorph of DCA.

FIG. 6 illustrates a DSC pattern of Form A polymorph of DCA.

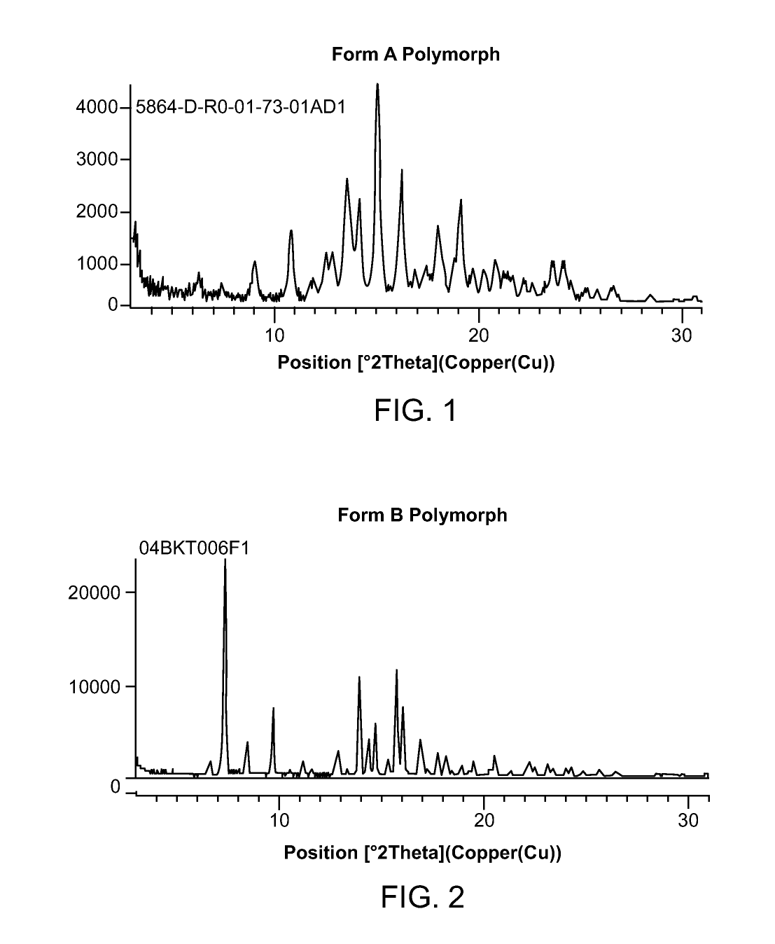

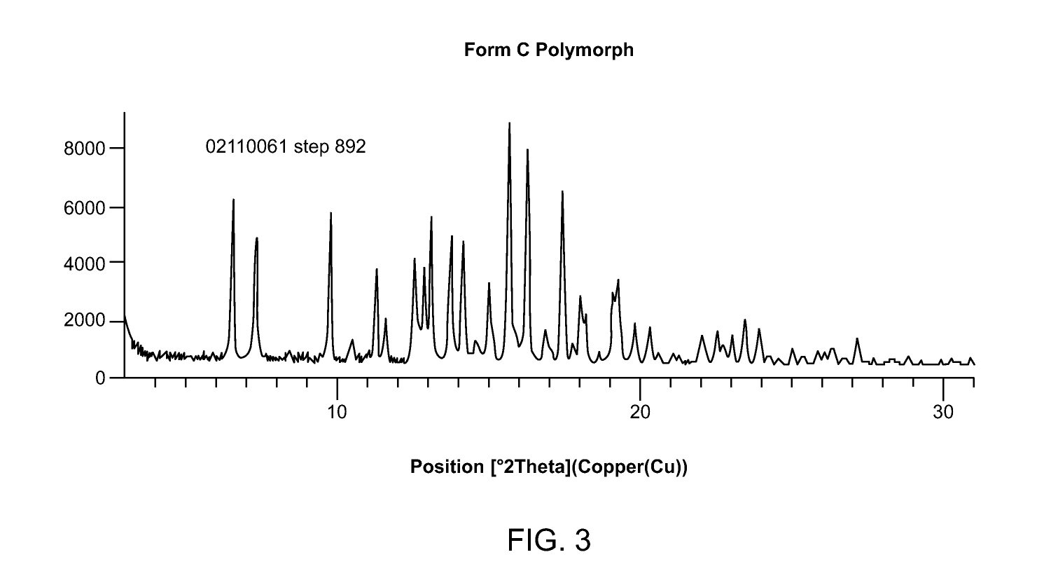

### DETAILED DESCRIPTION OF THE INVENTION

### Definition

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

As used herein, certain terms may have the following defined meanings. As used in the specification and claims, the singular form “a,” “an” and “the” include singular and plural references unless the context clearly dictates otherwise. Thus, for example, reference to “a solvent” includes a plurality of the same or different solvents.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations. Each numerical parameter should at least be construed in light of the number of reported significant digits and by applying ordinary rounding techniques. In certain instances, as will be apparent to the skilled artisan, the “about” when used before a numerical designation, e.g., temperature, time, amount, and concentration, including range, indicates approximations which may vary by (+) or (−) 10%, 5% or 1%.

As used herein, the term “comprising” is intended to mean that the compounds, compositions, processes, and methods include the recited elements, but not exclude others. “Consisting essentially of” when used to define compositions and methods, shall mean excluding other elements of any essential significance to the compounds, compositions, processes, or methods. “Consisting of” shall mean excluding more than trace elements of other ingredients for claimed compounds or compositions and substantial process or method steps. Embodiments defined by each of these transition terms are within the scope of this invention. Accordingly, it is intended that the processes methods, compositions and compounds can include additional steps and components (comprising) or alternatively include additional steps and compounds or compositions of no significance (consisting essentially of) or alternatively, intending only the stated steps or compounds or compositions (consisting of).

As used herein, the numbering of the steroidal scaffold and the rings in it, follows the general convention:

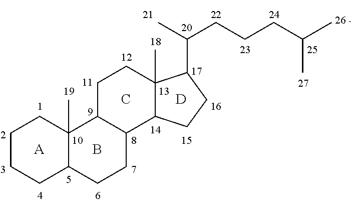

It is to be understood that unless otherwise specified, the scaffolds only represents the position of carbon atoms. One or more bonds between two adjacent carbon atoms may be a double bond and one or more of carbon atoms be may optionally substituted.

The term “Δ(or delta)-9,11-ene steroidal” or “Δ-9,11-ene compound” as used herein refers to a steroidal compound having a double bond between the 9 and 11 carbon atoms which is represented by the scaffold of:

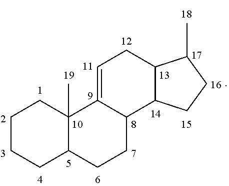 [SMILES: CC1CCC2C3CCC4CCCCC4(C)C3=CCC12]

As used herein, even without specific designation, the stereochemistry at the B, C, D ring junctions is that most commonly found in natural steroids, i.e.:

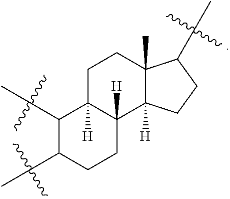

The term “2 carbon olefination reagent” refers to an olefination reagent that replaces the oxygen of a keto group with a Me-CH=moiety.

The term “acid” refers to regents capable of donating H⁺ or to “Lewis acids” that are electron pair acceptors. Lewis acids include oraganometallic reagents such as alkyl aluminum halides (e.g. Et₂AlCl and MeAlCl₂).

The term “alkoxy” refers to —O-alkyl, where alkyl is as defined above. Non-limiting examples include, methoxy, ethoxy, isopropoxy, propoxy, tertiary butoxy, isobutoxy, butoxy, and the likes.

The term “alkyl” refers to monovalent saturated aliphatic hydrocarbyl groups having from 1 to 10 carbon atoms (i.e., C₁-C₁₀ alkyl) or 1 to 6 carbon atoms (i.e., C₁-C₆ alkyl), or 1 to 4 carbon atoms. This term includes, by way of non-limiting example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “allylic oxidation” refers to oxidizing the alpha position of a double bond, preferably by incorporating one or more of a hydroxy, —OOH, —OO-alkyl, and oxo group at that alpha position. Preferably, such oxidation incorporates a hydroxy, and more preferably, an oxo group.

The term “aryl” refers to a monovalent, aromatic ring having 6-10 ring carbon atoms. Examples of aryl include phenyl and napthyl.

The term C_{X}, wherein x is an integer, when placed before a group, refers to that group containing x carbon atoms.

The term “dehydrating condition” refers to a condition under which hydroxy group and a hydrogen atom in an adjacent carbon atom is removed to provide an alkene. Dehydration conditions also include converting the hydroxy group to a leaving group such as chloro, bromo, tosylate, mesylate, triflate, or —OS(O)Cl. Dehydration or dehydrating is accomplished, for example by a dehydration reagent or simply by heating. Such non-limiting conditions include treatment with an acid, thionyl chloride, at the like.

The term “halo” refers to fluoro, chlroro, bromo, and/or iodo

The term “hydrogenation conditions” refers to conditions and catalysts for introducing H₂ across one or more double bonds, preferably using a hydrogenation catalyst. Hydrogenation catalysts include those based on platinum group metals (platinum, palladium, rhodium, and ruthenium and their oxides and hydroxides) such as Pd/C and PtO₂.

The term “hydroxy protecting group” refers to a group capable of protecting the hydroxy (—OH) group of a compound and releasing the hydroxy group under deprotection conditions. Common such groups include acyl (which forms an ester with the oxygen atom of the hydroxy group), such as acetyl, benzoyl, and groups that form an ether with the oxygen atom of the hydroxy group, such as methyl, allyl, propargyl, benzyl, methoxybenzyl, and methoxymethyl, silyl ethers, etc. Hydroxy protecting groups are well known in the field of organic synthesis. Suitable, non-limiting hydroxy protecting groups and other protecting groups which may be employed according to this invention, and the conditions for their deprotection, are described in books such as Protective groups in organic synthesis, 3 ed., T. W. Greene and P. G. M. Wuts, eds., John Wiley & Sons, Inc., New York, N.Y., U.S.A., 1999, and in its later editions, and will be well known to a person of ordinary skill in the art, which is incorporated by reference in its entirety.

The term “olefination reagent” refers to a regents that perform olefination, i.e., react with ketones to form olefins. The term “olefin forming conditions” refers to conditions to carry out such transformations. Examples of such reagents include Wittig and Wittig Horner reagents and examples of such conditions incude Wittig and Wittig Horner olefination conditions.

The term “ketal” refers to a group having two —OR^{x} groups attached to the same carbon atom in a molecule, where R^{x} represents a hydrocarbyl group. As is well known to the skilled artisan, ketals are susceptible to acidic hydrolysis under mild conditions in aqueous acids.

The term “oxidizing” with respect to a molecule refers to removing electrons from that molecule. In this way, for example, oxygen can be added to a molecule or hydrogen can be removed from a molecule. Oxidizing is effected, e.g., by oxidizing agents and electrochemically. The term “oxidizing conditions” refers to suitable conditions for oxidizing a molecule including microbial oxidation as disclosed herein.

The term “oxidizing agent” refers to a reagent which is capable of oxidizing a molecule, and include, without limitation, “chromium oxidizing agents” and “copper oxidizing agents”. In this way, oxygen can be added to a molecule or hydrogen can be removed from a molecule. Oxidizing agents include by way of example only dioxirane, ozone, di-^{t}butyltrioxide, oxygen, chloranil, dichlorodicyanobezoquinone, peracids, such as percarboxylic acids, Jones reagent, alkyl hydroperoxides, such as tertiary-butyl hydroperoxide (optionally used with CuI and a hypochlorite), hypochlorite, pyridinium chlorochromate, CrO₃, and Cu (II) or Cu (III) compounds, or mixtures thereof. More than one oxidizing agents may be used together for oxidizing a compound, where one of the oxidizing agents, preferably the metal-containing oxidizing agent, such as a chromium or a copper oxidizing agent, may used in a catalytic amount. A preferred oxidizing agent is a hydroperoxide and a cuprous salt, such as tertiary butyl hydroperoxide and CuI.

The term “pharmaceutically acceptable” refers to safe and non-toxic for in vivo, preferably for human, administration.

The term “pharmaceutically acceptable salt” or “salt thereof” refers to pharmaceutically acceptable salts of DCA, which salts are derived from a variety of organic and inorganic counter ions well known in the art and include, by way of example only, sodium, potassium, calcium, magnesium, ammonium, and tetraalkylammonium.

The term “reducing” refers to addition of one or more electrons to a molecule, and for example, allowing hydrogen to be added to a molecule and include hydrogenation conditions. The term “reducing agent” refers to a reagent which can donate electrons in an oxidation-reduction reaction, and, for example, allowing hydrogen to be added to a molecule. The term “reducing conditions” refers to suitable conditions, including hydrogenation conditions, for allowing electron and/or hydrogen to be added to a molecule. Suitable reducing agents include, without limitation, lithium, sodium, potassium, aluminum amalgam, lithium aluminum hydride, sodium borohydride, sodium cyanoborohydride, lithium tri-^{t}butoxy aluminum hydride, di^{t}butoxy aluminum hydride, lithium triethyl borohydride and the like.

The various starting materials, intermediates, and compounds of the preferred embodiments may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

Certain non-limiting examples of compounds, compositions, and processes of this invention are schematically illustrated below.

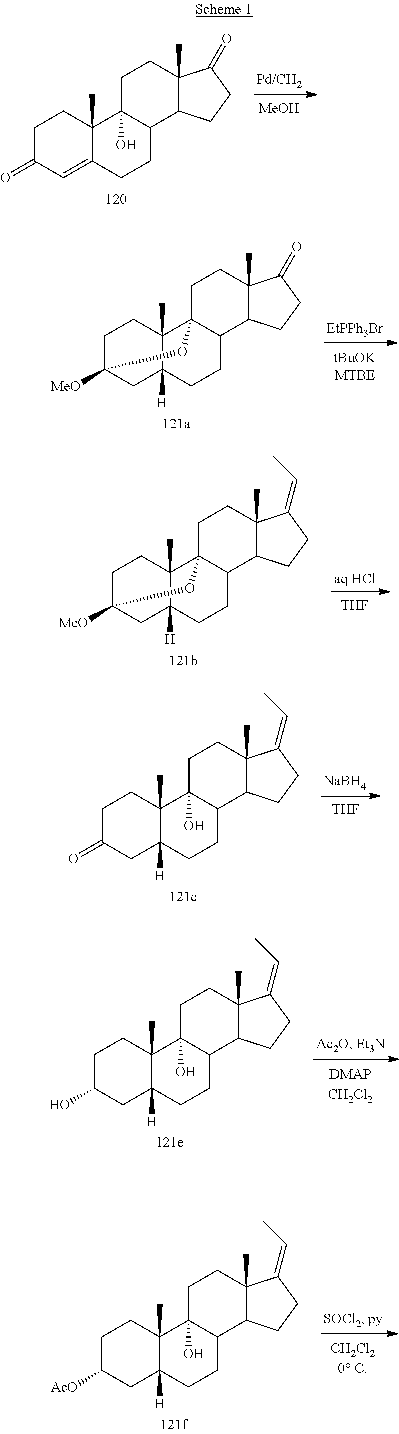

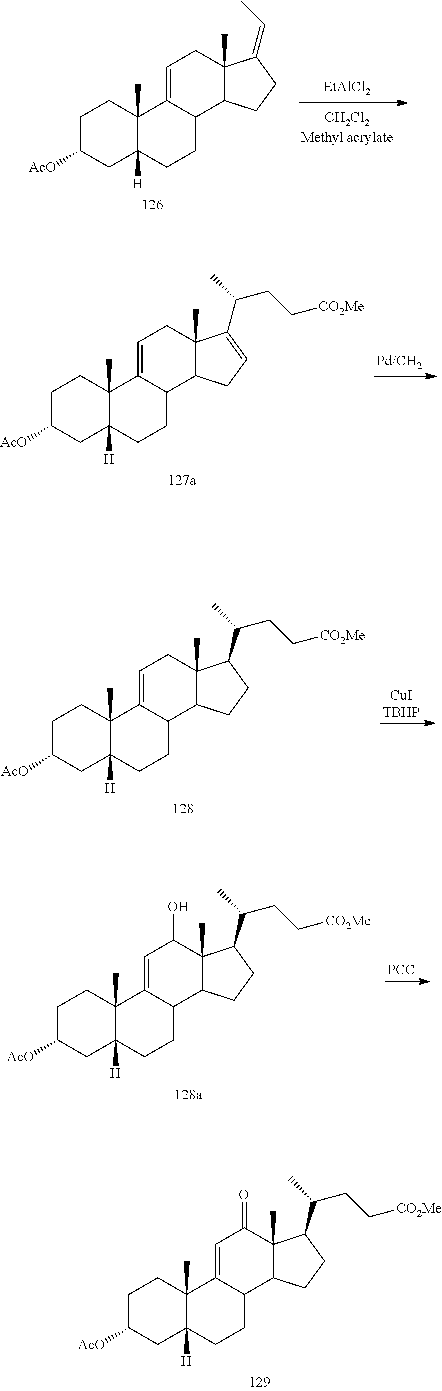

Compound 121a (obtained from hydrogenation of 120 in methanol) undergoes Wittig reaction n to give crude 121b (typically 55-68% 121b with around 1% E-isomer and 35-48% phosphorus-containing impurities). Acetic acid extractive purification of the product gave 121b (101% as is yield, purity 90.8% (area under the curve of the corresponding high performance liquid chromatogram (HPLC), or simply AUC) with 1.9% E-isomer and 5.5% phosphorus-containing impurities). Silica gel purification of the product gave 121b (120% as is yield, purity 90.8% (AUC) with 1.9% E-isomer and 5.1% phosphorus-containing impurities). Use of a more hindered base, 2,6-lutidine instead of pyridine, resulted in a much slower dehydration to form 126 (less than 9% conversion after 6.5 hours at 0° C.; 43% conversion after 15 hours at ambient temperature but with many impurities). In order to remove the magnesium sulfate drying of the 121f solution in dichloromethane, prior to the dehydration step, it was demonstrated that additional thionyl chloride (0.3 equiv) drove the reaction to completion (with 1.1 equivalents the reactions contained 15-19% unreacted 121f; adding 0.3 equivalents the reactions contained no unreacted 121f and had typical reaction profiles).

On a 5-g scale the ene reaction on 126 under standard conditions gave 127a [5.95 g, 95.0%, 90.3% (AUC), by GC-MS, PCI lot # D-170-190a] as a viscous liquid. This was hydrogenated at 23 psi hydrogen pressure under standard conditions to give, after work up, crude 128 [5.5 g, 92%, 84.5% (AUC) by GC-MS] as a white solid.

Residual metal analysis of a sample of recrystallized 129 showed 2 ppm Cu and 81 ppm Cr; therefore additional steps for metal remediation are not contemplated. Reduction in copper iodide loading (from 0.7 equiv to 0.35 equiv) in acetonitrile at 50° C. with TBHP (2.5 equiv) resulted in the oxidation taking too long (48 hours to reach completion compared with 17 hours). A 20-g oxidation was carried out; after quenching with sodium bisulfite solution and washing with brine a still water-wet solution of 128a/129b in acetonitrile was obtained. This was used to test direct oxidation of the product in this solution in an effort to reduce the processing. Reactions with PCC and activated MnO₂ gave no oxidation; with oxone, the major product was a new compound (by HPLC) instead of 129. When the acetonitrile solution was dried, the PCC was successful but the activated MnO₂ and oxone reactions gave no reaction (by TLC). 129 gave a good dose-response curve using CAD. 128 and 128a are both detectable using the CAD system (RRT 1.85 and 1.36); 128a showed as a double peak possibly due to epimers of the alcohol.

According to the aspects related to the stereoselective reduction of steroid dienes to provide DCA, illustrative compositions and methods of this invention are schematically illustrated below using CH₃CO— as the R¹CO— group. A variety of R¹ and R² (see above) groups can be employed in accordance with invention and based on synthetic methods known to the skilled artisan. See for example, PCT application publication no. WO 2011/075701 and U.S. patent application publication no. 2008/0318870, each of which is incorporated herein in its entirety by reference.

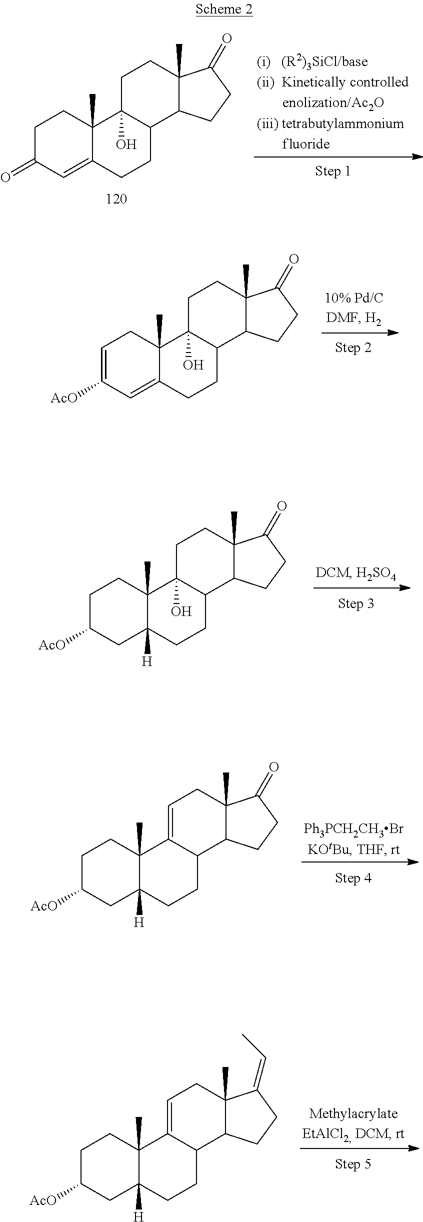

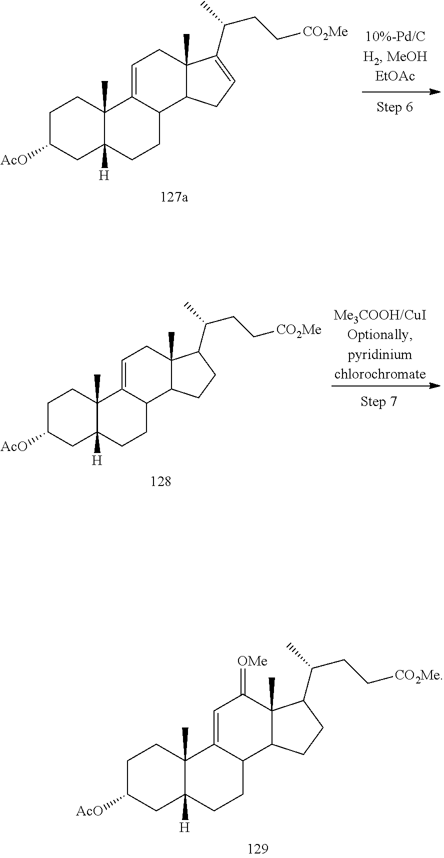

As will be apparent to the skilled artisan, the 17-keto group may be protected, for example, as a ketal, while Step 1 is performed and subsequently deprotected. For performing Step 1, the following methods and reagents can also be used

For example, any orthogonal protecting group that can be cleaved in the presence of an acetate/ester functionality. Illustrative examples include, certain benzyl type protecting groups, other silyl protecting groups, and acetal protecting groups. It is also contemplated that the kinetically controlled enolization can be performed without protecting the tertiary C-9 alcohol. Also, the selection of the protecting group could determine if a separate deprotection is needed (i.e. step (iii) below). If a benzyl type group is used, then this group would be removed during hydrogenation, which is the next step in the synthesis.

The enolization could be done with a variety of kinetic bases like LDA, Na or KHMDS, etc. It is also contemplated that bases like pyridine, triethyl amine, morpholine, Hunig's base, carbonate bases, hydroxides (depending if the C-9 alcohol is protected or not), etc. in the presence of Ac₂O or AcCl can provide the desired product.

In general, any reagent including a fluoride anion (F⁻) can be used. Fluoride is used for deprotecting a silicon based protecting group. If one of the other protecting groups mentioned above are used then other deprotection reagents would be needed. Hydrogenation, acid, or nothing (if the C-9 alcohol wasn't protected in the first place) are other possible reagents depending on the protecting group.

For performing the last step, Step 7, the following methods and reagents can also be used: TEMPO/bleach, TEMPO/Oxone, Pd/C & peroxides, peroxides, MnO₂ and PCC, SeO₂ and PCC, MnO₂ and another oxidant, SeO₂ and another oxidant, bleach and tBuOOH, Cr oxidants, etc, as are well known to the skilled artisan. If one proceeds via a 12-hdroxy allylic alcohol, then the 12-hydroxy group can be oxidized following a variety of well known reagents and methods.

As will be apparent to the skilled artisan, the solvents employed in the schemes above are illustrative and other solvents well known to the skilled artisan can also be used.

### EXAMPLES

In the examples below and elsewhere in the specification, the following abbreviations have the indicated meanings. If an abbreviation is not defined, it has its generally accepted meaning.

| | |
|---|---|
| Ac | Acetyl |
| DCA | Deoxycholic acid |
| DCM (CH₂Cl₂) | Dichloromethane |
| ELSD | Evaporative light scattering detection |
| EtOH | Ethanol |
| EtOAc | Ethyl acetate |
| G | Grams |
| GC-MS | Gas chromatography-mass specotrometry |
| H or h | Hour |
| HCl | Hydrochloric acid |
| HPLC | High pressure liquid chromatography |
| Hz | Hertz |
| HMDS | Hexamethyldisilazide |
| LiAl(O^{t}Bu)₃H | Lithium tri-tert-butoxyaluminum hydride |
| LOD | Loss on drying |
| Me | Methyl |
| MeOH | Methanol |
| MHz | Megahertz |
| Min | Minutes |
| mL | Milliliter |
| Mmol | Millimole |
| Mol | Mole |
| Na₂SO₄ | Sodium sulfate |
| NaOH | Sodium hydroxide |
| NMT | Not more than |
| Pd/C | Palladium on carbon |
| PtO₂ | Platinum oxide |
| TEMPO | 2,2,6,6-tetramethylpiperidine-N-oxyl |
| TFA | Trifluoroacetic acid |
| THF | Tetrahydrofuran |
| TLC | Thin layer chromatography |
| UV | Ultraviolent |
| Wt | Weight |

General:

All manipulations of oxygen- and moisture-sensitive materials were conducted with standard two-necked flame dried flasks under an argon or nitrogen atmosphere. Column chromatography was performed using silica gel (60-120 mesh). Analytical thin layer chromatography (TLC) was performed on Merck Kiesinger 60 F₂₅₄ (0.25 mm) plates. Visualization of spots was either by UV light (254 nm) or by charring with a solution of sulfuric acid (5%) and p-anisaldehyde (3%) in ethanol.

Apparatus:

Proton and carbon-13 nuclear magnetic resonance spectra (¹H NMR and ¹³C NMR) were recorded on a Varian Mercury-Gemini 200 (¹H NMR, 200 MHz; ¹³C NMR, 50 MHz) or a Varian Mercury-Inova 500 (¹H NMR, 500 MHz; ¹³C NMR, 125 MHz) spectrometer with solvent resonances as the internal standards (¹H NMR, CHCl₃ at 7.26 ppm or DMSO at 2.5 ppm and DMSO—H₂O at 3.33 ppm; ¹³C NMR, CDCl₃ at 77.0 ppm or DMSO at 39.5 ppm). ¹H NMR data are reported as follows: chemical shift (δ, ppm), multiplicity (s=singlet, d=doublet, t=triplet, q=quartet, br=broad, m=multiplet), coupling constants (Hz), and integration Infrared spectra (FT-IR) were run on a JASCO-460⁺ model. Mass spectra were obtained with a Perkin Elmer API-2000 spectrometer using ES⁺ mode. Melting points were determined using a LAB-INDIA melting point measuring apparatus and are uncorrected. HPLC chromatograms were recorded using a SHIMADZU-2010 model with a PDA detector. Specific optical rotations were determined employing a JASCO-1020 at 589 nm and are uncorrected.

DSC, TGA, XRPD and DVS data can be and were collected using the following instruments and procedures.

| Instrument | Vendor/Model# |
|---|---|
| Differential Scanning Calorimeter | Mettler 822^{e} DSC |
| Thermal Gravimetric Analyzer | Mettler 851^{e} SDTA/TGA |
| X-Ray Powder | CubiX-Pro XRD |
| Diffraction System | |
| Moisture-Sorption Analysis | Hiden IGAsorp Moisture Sorption |
| | Instrument |

### Differential Scanning Calorimetry Analysis (DSC)

DSC analyses were carried out on the samples “as is”. Samples were weighed in an aluminum pan, covered with a pierced lid, and then crimped. Analysis conditions were 30° C. to 200-350° C. ramped at 10° C./min.

### Thermal Gravimetric Analysis (TGA)

TGA analyses were carried out on the samples “as is.” Samples were weighed in an alumina crucible and analyzed from 30° C. to 200-350° C. and at a ramp rate of 10° C./min.

### X-Ray Powder Diffraction (XRPD)

Samples were analyzed “as is”. Samples were placed on Si zero-return ultra-micro sample holders. Analysis was performed using a 10 mm irradiated width and the following parameters were set within the hardware/software:

X-ray tube: Cu KV, 45 kV, 40 mA

### Detector: X′Celerator

### ASS Primary Slit: Fixed 1°

Divergence Slit (Prog): Automatic-5 mm irradiated length
Soller Slits: 0.02 radian
Scatter Slit (PASS): Automatic-5 mm observed length

### Scan Range: 3.0-45.0°

### Scan Mode Continuous

### Step Size: 0.02°

### Time per Step: 10 s

### Active Length: 2.54°

Following analysis, the data were converted from adjustable to fixed slits using the X′Pert HighScore Plus software with the following parameters:

### Fixed Divergence Slit Size: 1.00°, 1.59 mm

Dynamic Vapour Sorption (DVS)

Moisture-sorption experiments were carried out on 10-15 mg of material at 25° C. by performing an adsorption scan from 40 to 90% RH in steps of 10% RH and a desorption scan from 85 to 0% RH in steps of −10% RH. A second adsorption scan from 10 to 40% RH (at 25° C.) was performed to determine the moisture uptake from a drying state to the starting humidity. The sample was allowed to equilibrate for four hours at each point or until an asymptotic weight was reached. After the isothermal sorption scan, samples were dried at 60° C. at 0% RH for four hours to obtain the dry weight. XRPD analysis following moisture sorption and drying was performed to determine the solid form of the material.

Chemicals:

Unless otherwise noted, commercially available reagents were used without purification. Diethyl ether and THF were distilled from sodium/benzophenone. Laboratory grade anhydrous DMF, commercially available DCM, ethyl acetate and hexane were used.

### Example 1

### Characterization and Stability of Crystalline DCA Polymorphs

### A. Drying Experiments

Conversion of Form C to Form B was evaluated at 40° C. under vacuum. Two different lots of 215 mg and 134 mg of Form C were dried under vacuum at 40° C. After 2 hours, XRPD analysis indicated that both materials were converted to Form B. Karl Fisher analysis of post-drying material showed less than 0.1% water. Another Form C lot was dried under vacuum at 40° C. for 18 hours and XRPD analysis showed complete conversion to Form B.

TGA analysis of Form C indicated that 40° C. was not an optimum drying temperature and a higher drying temperature of 50° C. speeded up the drying and form conversion. One concern with higher drying temperature was the stability of Form B. However, the Form B crystals were surprisingly stable to prolonged heating at up to 70° C. To evaluate the stability of DCA Form B at 50° C. and 70° C., two lots of Form C were dried at 50° C. and 70° C. for 2 hours. XRPD analysis indicated form conversion to Form B was complete. The samples were dried further for 24 hours and retained for HPLC analysis. HPLC analysis showed no degradation after drying for 24 hours.

Another drying study was performed to evaluate the stability of Form B DCA dried at 50° C. with deionized water (DI-water) and EtOH. Samples of DCA (2.0 g) were combined with DI water and EtOH. The samples were then dried under vacuum for an extended period of time at 50° C. The samples were assayed by HPLC and the results demonstrated that Form B DCA was stable when dried in the presence of EtOH and water.

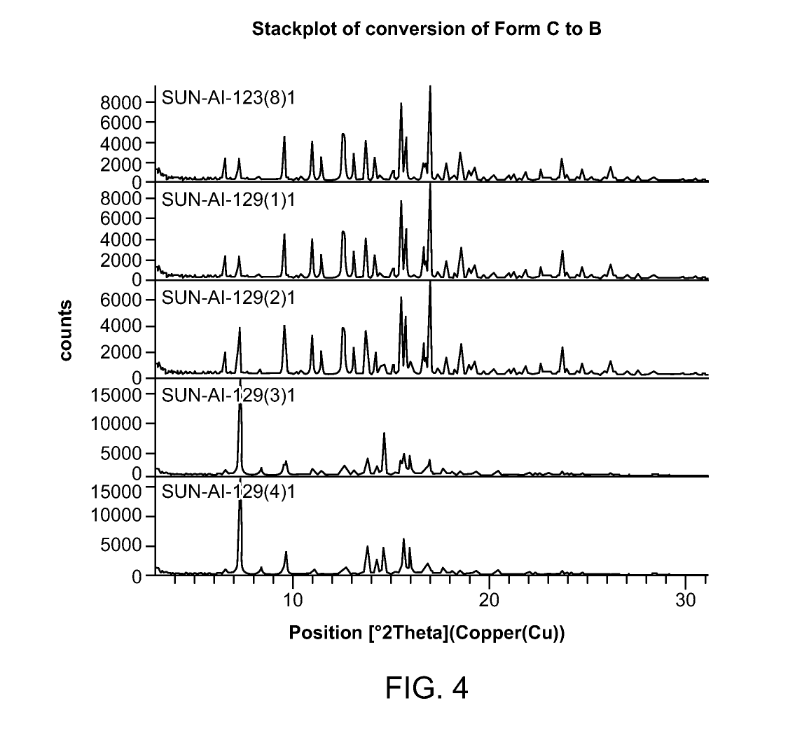

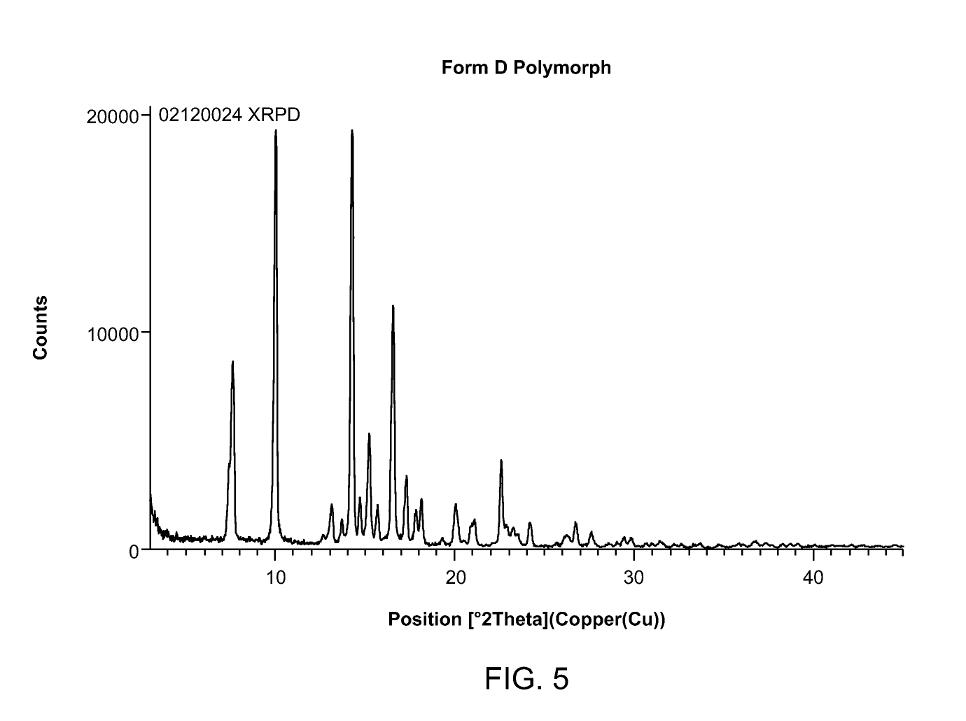

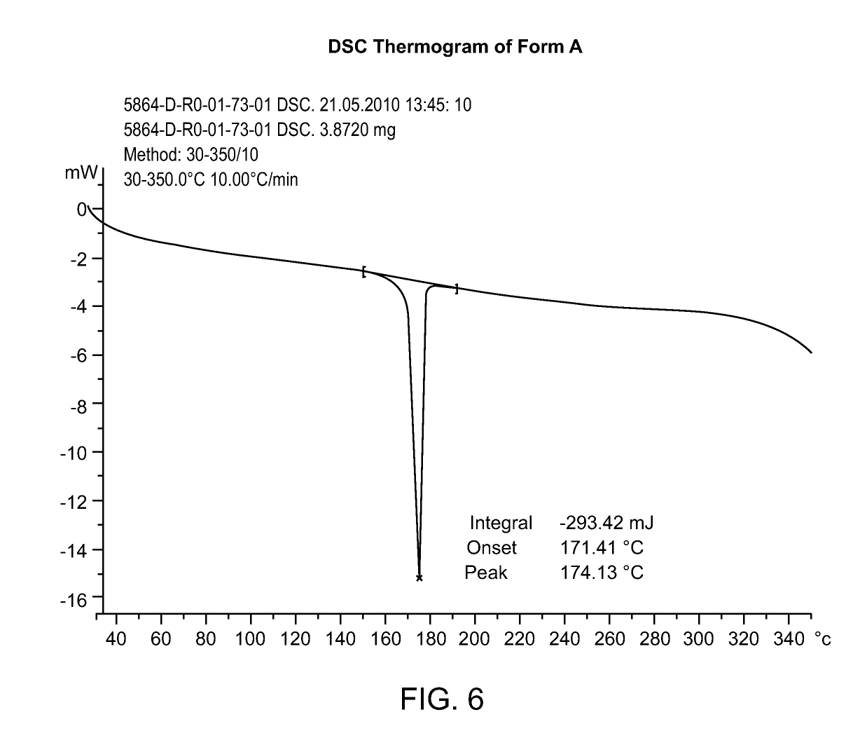

KF and XRPD analysis of the samples from drying study showed that anhydrate Form B contained less than 0.9% of water and hydrate Form C contained more than 1.9% of water. The form conversion of Form C to Form B at approximately 45° C. under vacuum was analyzed by XRPD every 20 minutes. FIG. 4 graphically illustrates the conversion of the Form C to Form B upon heating.

### B. Slurry Stability

To evaluate form stability under slurry conditions, Forms A and B were slurried in about 1:1.2 v/v EtOH/water at ambient temperature and at 50° C. Surprisingly, at ambient temperature, Form B, did not show any form conversion by XRPD; slurrying at 50° C. afforded Form C after 2 hours.

### C. Humidity Stress

Approximately 15 mg of Form B lot was stored at 95% relative humidity (RH) at ambient temperature. Even after 10 days, XRPD analysis showed no conversion to Form C. This surprising humidity/temperature stability of Form B was further evidenced from the following experiments. Form B samples were stored at 95% relative humidity (RH) and ambient temperature, and at 75% RH and 40° C. Even after 11 days, XRPD indicated no form conversion. KF showed increase of water content at variable degree depending on lots and storage conditions. The increase of water content appeared to reach a plateau after an initial water sorption period.

### D. Form C Preparation

A baseline crystallization was performed on 0.15 g scale following the current plant procedure. Thus, 148 mg of DCA Form B was dissolved in EtOH (1.57 mL) and water (0.178 mL), polish-filtered and added to water (4.44 mL). Residual DCA solution was rinsed with EtOH (0.4 mL) and water (0.044 mL) and added to the reaction. The resulting slurry was stirred at ambient temperature for 16 hours and filtered, affording 140 mg of solid, which was analyzed by XPRD without drying and found to be Form C.

### E. Form Conversions

### Conversion of Form B to Form C Via Slurry Experiment:

Approximately 217 mg of DCA Form B was mixed with 1.5 mL of EtOH/water (1:2.37 v/v). The mixture was heated at 50° C. with stirring for 2 hours and an aliquot was filtered to isolate wet solids for XRPD analysis. A sample was isolated after 4 hours and XRPD showed the material remained to be Form B.

### Conversion of Form B to Form A Via Heating:

20 mg of DCA Form B was weighed in an alumina crucible and heated from 30° C. to 150° C. at a ramp rate of 10° C./min and then held at 150° C. for 30 minutes. The material was cooled to ambient temperature rapidly on the instrument and analyzed by XRPD. XRPD results showed complete conversion to Form A.

### Conversion of Form B to Form D Via Heating:

19 mg of DCA Form B was weighed in an alumina crucible and heated from 30° C. to 135° C. at a ramp rate of 10° C./min and then held at 135° C. for 30 minutes. The material was cooled to ambient temperature rapidly on the instrument and analyzed by XRPD. XRPD results showed complete conversion to Form D.

### Example 2

### Preparation of Compound 126 from Compound 120 Via Ketal 121a

### A. Synthesis of 121a

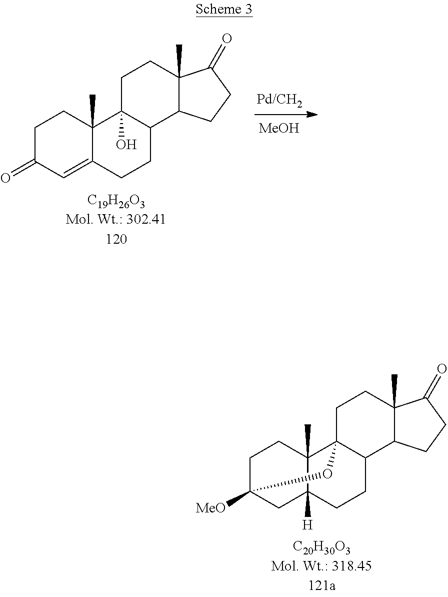

The hydrogenation was performed in a 150-g scale. Hydrogenation was complete with 3 hours and the hydrogen atmosphere replaced with nitrogen.

### B. Synthesis and Purification of 121b (Ref: Experiment D-168-165, D-168-167, D-168-174)

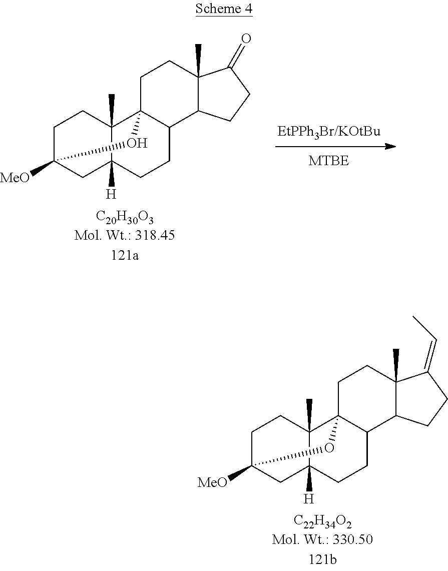 [SMILES: CO[C@@]12CC[C@@]3(C)[C@H](CCC4C5CCC(=O)[C@@]5(C)CC[C@@]43O1)C2 ; "MTBE" ; C/C=C1/CCC2C3CC[C@@H]4C[C@]5(OC)CC[C@]4(C)[C@]3(CC[C@]12C)O5]

The Wittig reaction in methyl tertiary butyl ether (MTBE) was repeated using the batch of 121a from the methanol based hydrogenation as a use-test of this material. In addition the three potential processes of removing the phosphorus-containing impurities (acetic acid or silica gel treatment of 121b, and crystallization of 121e instead) were compared.

Potassium tert-butoxide (5.29 g, 1.5 equiv) was added to a solution of ethyltriphenylphosphonium bromide (20.98 g, 1.8 equiv) in MTBE (60 mL) under N₂ atmosphere and the reddish orange solution was stirred at room temperature for 2.5 hours. A solution of 121a (10.0 g, PCI lot #-111) in MTBE (40 mL) was added over 5 minutes and the resulting reaction mixture was stirred at room temperature for 17.5 hours at which point the reaction was deemed complete by GC-MS analysis with a ratio of 98.4:1.6 121b:E-isomer (see Table 1).

The reaction mixture was filtered through a Buchner funnel and the filter cake washed with MTBE (3×100 mL). After evaporation to dryness, the residue was dissolved in heptanes (200 mL), charged with glacial acetic acid (50 mL) and agitated vigorously. Water (25 mL) was added to separate the layers and the organic layer washed with water (50 mL) to remove any remaining acetic acid. After concentration, 121b [10.50 g, 101%, 90.8% (AUC) by GC-MS, containing 1.9% (AUC) of the isomer, PCI lot # D-168-165e] was isolated.

If the acetic acid purification were to be chosen (instead of purifying at 121e) it would be expected that an extra acetic acid extraction of the heptane layer would be able to remove all the phosphorus-containing impurities.

TABLE 1
Wittig Reaction in MTBE (Ref D-168-165)

| | %(AUC) by GC-MS | | | | | |
|---|---|---|---|---|---|---|
| | A | B | I | 121b | 121a | C |
| | 12.42 | 12.90 | 13.74 | 13.88 | 14.06 | 14.45 |
| Sample | min | min | min | min | min | min |
| 1.0 h | 10.9 | nd | 0.8 | 45.3 | 8.6 | 34.4 |
| 2.0 h | 10.1 | nd | 0.9 | 47.2 | 4.2 | 37.6 |
| 17.5 h | 8.6 | nd | 1.0 | 62.6 | 0.2 | 27.7 |
| D-168-165c | 0.6 | 0.1 | 1.9 | 90.8 | 0.2 | 4.8 |

Note:
I is the presumed E- isomer of 121b.
Note:
A, B, C are phosphorus-containing impurities.

The Wittig reaction was repeated on 10-g scale but using MTBE/heptane (1:1) as the solvent system. This would allow the purification via silica slurry to be carried out without any solvent swap at the end of the Wittig reaction prior to purification thus making the process more streamlined. When the reaction was complete, the mixture was filtered through a Buchner funnel and the filter cake was washed with 1:1 MTBE/heptanes (3×100 mL). Silica gel (20 g) was added to the combined filtrate, stirred for 3 hours and then removed by filtration, washing the this filter cake with 1:1 MTBE/heptanes (3×100 mL). After concentration 121b [12.47 g, 120% (solvent wet), 90.8% (AUC) by GC-MS, containing 2.2% (AUC) of the isomer, PCI lot # D-168-167c] was obtained as an oil (see Table 2). The overall level of phosphorus-containing impurities was similar to the acetic acid purification [5.1% versus 5.5% (AUC) by GC-MS].

TABLE 2
Wittig Reaction in MTBE/heptanes (Ref D-168-167)

| | %(AUC) by GC-MS | | | | | |
|---|---|---|---|---|---|---|
| | A | B | I | 121b | 121a | C |
| | 12.42 | 12.90 | 13.74 | 13.88 | 14.06 | 14.45 |
| Sample | min | min | min | min | min | min |
| 1.0 h | 11.1 | nd | 1.1 | 46.5 | 16.6 | 24.8 |
| 2.0 h | 9.8 | nd | 1.2 | 54.5 | 9.4 | 25.1 |
| 17.5 h | 8.6 | nd | 1.2 | 49.2 | 0.1 | 40.9 |
| D-168-167c | 2.1 | 0.2 | 2.2 | 90.8 | 0.4 | 2.8 |

During the addition of 121a to the ylide an 8° C. exotherm was observed. At reaction completion a ratio of 98.2:1.7 121b:E-isomer was obtained (see Table 3). The reaction mixture was filtered through a Buchner funnel and the filter cake was washed with MTBE (3×500 mL). The filtrate was concentrated to give crude 121b [90.64 g, 175%, 68.6% (AUC) by GC-MS, PCI lot # D-168-174c]. This crude was not purified any further but taken directly into the hydrolysis step.

TABLE 3
50 g Wittig Reaction in MTBE (Ref D-168-174)

| | %(AUC) by GC-MS | | | | | |
|---|---|---|---|---|---|---|
| | A | B | I | 121b | 121a | C |
| | 12.42 | 12.90 | 13.74 | 13.88 | 14.06 | 14.45 |
| Sample | min | min | min | min | min | min |
| 1.0 h | 10.3 | nd | 0.8 | 41.8 | 11.4 | 35.7 |
| 17.5 h | 8.3 | nd | 0.9 | 54.4 | 0.1 | 35.6 |
| D-168-174c | 7.7 | nd | 1.1 | 68.6 | 0.1 | 22.0 |

C. One-Pot Synthesis of 121e from 121b (Ref: Experiment D-168-171, D-118-178)
Direct Synthesis of 121e from 121b

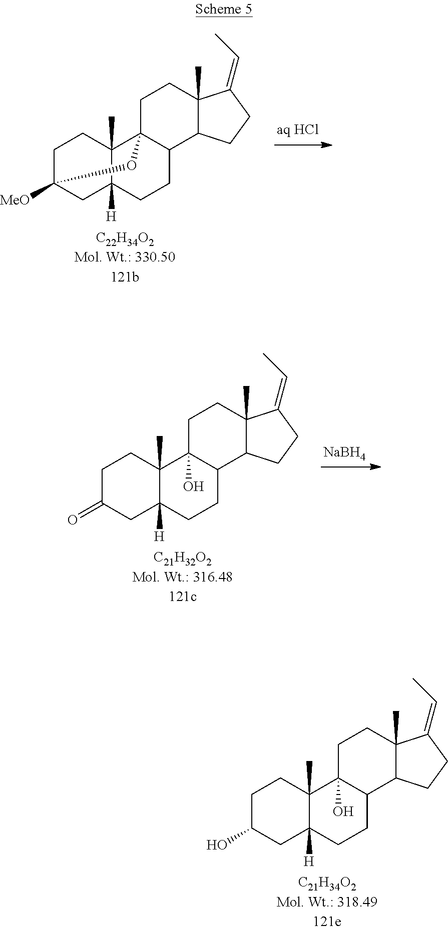

The one-pot synthesis of 121e from 121b was investigated using fewer equivalents of sodium borohydride and replacing methanol with water as co-solvent in an attempt to streamline the work up.

A portion of a heptane/acetic acid purified 121b [20.0 g, 86.1% (AUC) by GC-MS, PCI lot # D-168-162a] was stirred with THF (15 volumes, 300 mL) and 2 M HCl (5 volumes, 100 mL) at ambient temperature. Although 5.4% (AUC) of 121b remained after 16 hours, the reaction mixture was worked up being basified to pH 12 with 6 M NaOH. The organic layer was separated and returned to the reaction flask. Sodium borohydride (0.5 equiv, 1.14 g) was dissolved in basified water (1 volume, 20 mL, pH 10 using 6 M NaOH). Monitoring the reaction by TLC, it was approximately halfway complete after three hours (slower than when using methanol as co-solvent with 1.5 equivalents borohydride).

Additional sodium borohydride (0.5 equiv) was added and after stirring overnight the reaction was complete by TLC. The aqueous layer of the reaction mixture was separated and discarded after confirming by TLC that it contained no product. The organic layer was concentrated to dryness and then re-dissolved in MTBE (550 mL). This was washed with 1M hydrochloric acid (250 mL) and water (250 mL). The acid wash did not produce any hydrogen gas. Concentration of the organic layer followed by a methanol chase (100 mL) gave crude 121e (24.05 g) as a white, sticky solid which was recrystallized from methanol (120 mL) and water (22 ml) to give 121e [13.60 g, 71% from 121b, 96.1% (AUC) by RI HPLC, PCI lot # D-168-171e] as a white powder.

The hydrolysis step was run as previously but left over for 2 days before being worked up.

### D. Hydrolysis of 121b (Ref: Expt D-173-88)

A mixture of 121b (6.2 g, PCI lot D-168-167c), THF (50 mL), MTBE (50 mL) and 2 M HCl (50 mL) was stirred at ambient temperature for 24 hours; GC-MS indicated essentially no reaction. The reaction mixture readily separated into two layers when stirring stopped. The reaction mixture was then heated to reflux for 16 hours; GC-MS indicated an approximately 60:40 ratio of 121b:121c along with several isomers of both being formed. Use of MTBE/THF mixture for the hydrolysis does not appear to offer any advantage.

E. Synthesis of 126 from 121e

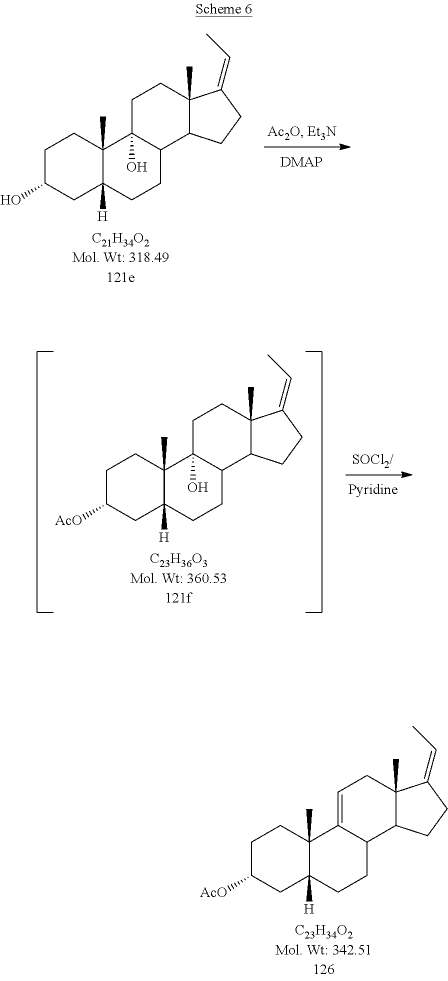

### F. Dehydration in Presence of DMAP to Promote Formation of Shoulder Peak Impurity (Ref: Experiment D-170-179)

In order to elucidate the structure of the impurity that is responsible for the shoulder peak in the GC-MS chromatogram, the direct synthesis of 126 from 121e using DMAP as the base was repeated on a 2.0-g scale. These conditions had previously produced 126 containing 7.6% (AUC) of the shoulder peak by GC-MS. This impurity is suspected to be the Δ-8 isomer of 126.

Dehydration in Presence of 2,6-Lutidine (Ref: Experiment D-170-184):

The effect of a more hindered aromatic base on the dehydration of 121f in dichloromethane to prepare 126 was examined. The experimental details are summarized as follows. A solution of 121f (0.25 g) in dichloromethane was treated with thionyl chloride (1.1 equiv) and 2,6-lutidine (3.5 equiv) at 0° C. The reaction was much slower as only 6.4% of 126 formed after 3.5 hours when compared to pyridine. Additional thionyl chloride (1.5 equiv) and 3.5 equivalents of 2,6-lutidine (3.5 equiv) did not increase the rate of dehydration significantly (8.6% of 126 formed after 6.5 hours). Allowing the reaction mixture to stir at ambient temperature did increase the rate of dehydration but was accompanied by formation of impurities. After 15 hours, the reaction mixture 42.9% (AUC) of 126 with 3.5% of the corresponding E isomer and 34.2% of 121f; the shoulder peak impurity was also present (1.7%). Therefore, under the conditions tested, 2,6-lutidine offers no advantage over pyridine as a base for the dehydration of 121f.

Synthesis of 126 without Mgso₄ Drying Step (Ref: Experiment D-170-191):

To eliminate the magnesium sulfate drying step prior to the dehydration of 121f solution in dichloromethane, the use of excess reagents in the dehydration steps (to compensate for any residual water) was examined. Acetylation of 121e (3.0 g) was performed using acetic anhydride (1.1 equiv), triethylamine (2.0 equiv) and DMAP (0.1 equiv) in dichloromethane (45 mL) at room temperature. After one hour, 121e was completely consumed and 95.8% (AUC) of 121f was detected by GC-MS. The reaction mixture was washed with water (25 mL), followed by 0.5 M HCl (25 mL), water (25 mL) and saturated brine solution (25 mL) and then split into two portions.

The first portion was treated with thionyl chloride (1.1 equiv) and pyridine (2.5 equiv) at 0° C. After 1.75 hours the reaction gave 71.9% (AUC) of 126 along with 19.0% (AUC) of 121f. Thionyl chloride (0.3 equiv) and pyridine (0.5 equiv) were added; after 0.75 hours the reaction was deemed complete with no 121f detected. The reaction contained 88.2% (AUC) of 126 with 4.0% (AUC) of the corresponding E isomer and 3.3% (AUC) of the shoulder peak.

The second portion was treated with thionyl chloride (1.1 equiv) and pyridine (3.0 equiv) at 0° C. After 2 hours, the reaction gave 76.7% (AUC) of 126 with 14.9% (AUC) of 121f. Thionyl chloride (0.3 equiv) was added and the reaction was complete within 1 hour with no 121f detected. The reaction contained 86.2% (AUC) of 126 formed along with 4.0% (AUC) of the corresponding E isomer and 3.4% (AUC) of the shoulder peak by GC-MS.

The dehydration can be made to go to completion using excess reagents added in during the course of the reaction.

### G. Ene Reaction on 126 to Prepare 127a (Ref: Experiment D-170-187)

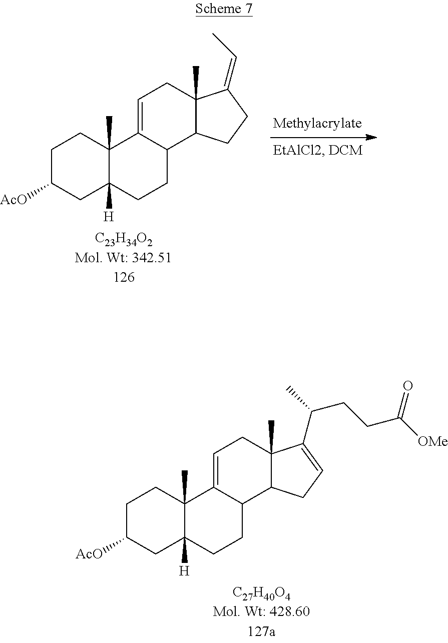

Methyl acrylate (2.38 equiv) was added over a period of 15 minutes to a solution of 126 (5.0 g) in dichloromethane (75 mL) at 0° C. under nitrogen atmosphere. After stirring the reaction mixture for 1 hour at 0° C., ethylaluminium dichloride (3.0 equiv, 1.8M solution in toluene) was charged over a period of 1 hour and the reaction mixture was stirred at ambient temperature. After 24 hours, 86.2% (AUC) of 127a was detected along with 1.9% of 126 by GC-MS. The reaction mixture was poured into ice water (200 mL) and extracted with dichloromethane (100 mL). The organic layer was washed with water (50 mL), saturated NaHCO₃ solution (50 mL), saturated brine solution (50 mL), and dried over anhydrous MgSO₄. The resulting solution was concentrated to obtain 10.0 g of the residue (D-170-190). The above residue was dissolved in hexane (50 mL) and passed through a silica bed, washed with 10% of EtOAc in hexane (200 mL). The filtrate was concentrated to obtain 5.95 g of [95.0%, 90.3% (AUC) by GC-MS-PCI lot # D-170-190a] 127a as a viscous liquid-used directly in the next reaction.

### H. Synthesis of 128 (Ref: Experiment D-170-197)

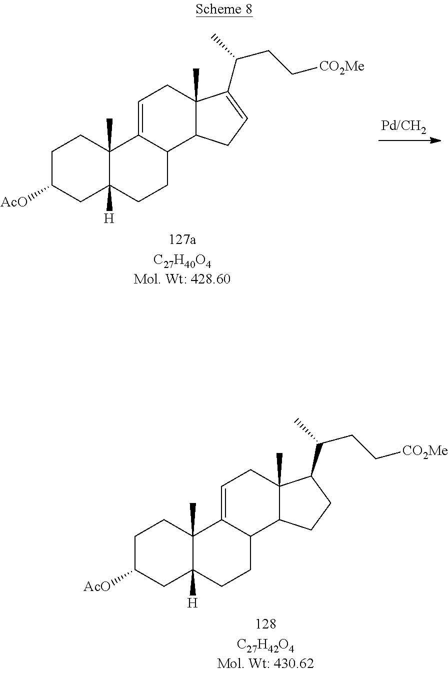 [SMILES: COC(=O)CC[C@@H](C)C1=CCC2C3CC[C@@H]4C[C@H](OC(C)=O)CC[C@]4(C)C3=CC[C@]12C ; COC(=O)CC[C@@H](C)[C@H]1CCC2C3CC[C@@H]4C[C@H](OC(C)=O)CC[C@]4(C)C3=CC[C@@]21C]

The hydrogenation was carried out as follows. A mixture of 127a (5.95 g), 10% palladium on carbon (0.6 g), ethyl acetate (34 mL) and methanol (16 mL) was hydrogenated at 23 psi for 16 hours when the reaction was deemed complete with 83% (AUC) of 128 was detected by GC-MS. The reaction mixture was filtered through Celite and washed with EtOAc (100 mL). The filtrate was concentrated to obtain 5.5 g (92.0%, 84.5% (AUC) by GC-MS) of crude 128 as a white solid.

### Example 3

### Allylic Oxidation of Compound 128

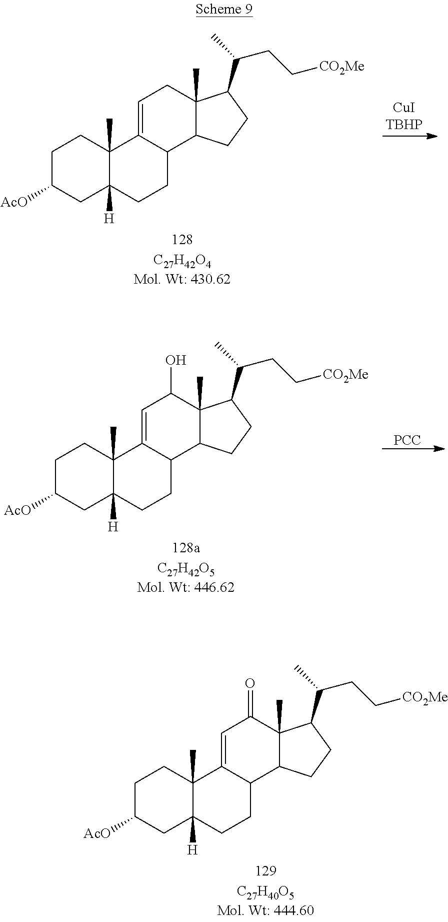

All the reactions reported below were monitored by HPLC (refractive index (RI) and UV methods) and were carried out using a new lot of 128.

A. Preparation of 128a

Oxidation with Reduced Copper Iodide Loading (Ref. Expt D-169-170)

Oxidation of 128 (2-g scale) was carried out using 2.5 equivalents TBHP at 50° C. but using only half the amount of copper iodide (0.35 equiv) compared with last week's reactions. The reaction was monitored for the consumption of 128. It was apparent that the reaction was slower and therefore it is recommended that the stoichiometry of copper iodide remain at 0.7 equivalents under these conditions

TABLE 4
Oxidation of 128 with reduced copper iodide loading

| | %(AUC) by HPLC (RI) | | |
|---|---|---|---|
| Time | 129 | 128a | 128 |
| 10 h | 6.3 | 63.2 | 24.4 |
| 24 h | 27.1 | 64.4 | 3.9 |
| 48 h | 36.2 | 57.3 | 1.8 |

B. Scale Up of Preparation of 128a

To prepare a batch of crude 128a for use in trial oxidations of the second stage, the oxidation of 128 was carried out as follows (Ref: Expt D-173-85). TBHP (16 ml, 2.5 equiv) was added in 10 equal portions over 9 hours to a mixture of 128 (20 g), copper iodide (6.0 g, 0.7 equiv) and acetonitrile (280 ml) at 50° C.; the reaction mixture was heated for an additional 7 hours. The cooled mixture was quenched with saturated sodium bisulfite (25 ml) and then washed with saturated brine (4×50 mL) to give an acetonitrile solution of crude 128a [lot D-173-85A, 61.7% (AUC) 128a, 29.8% 129 and 2.9% 128, KF ˜25%).

C. Test Oxidations of 128a

A series of oxidations was carried out on the crude 128a in acetonitrile. Typically 128a (˜0.3 g input based upon concentration of the wet acetonitrile solution) was treated with each oxidant (1 equiv) at ambient temperature for 16 hours. For reactions using dry acetonitrile, the solution of acetonitrile isolated in the previous experiment was concentrated to dryness and chased with acetonitrile to remove residual water before being redissolved in acetonitrile. PCC was found to work only on the dried acetonitrile solution (reactions monitored by TLC—not worked up). Activate manganese dioxide resulted in no reaction (as monitored by TLC). Oxone resulted in reaction under wet conditions but a new product formed which was the major component (presumably the wetness of the reaction conditions allows some oxone to dissolve and react). Therefore it may be possible to conduct the second oxidation in dry acetonitrile using PCC.

TABLE 5
Results of oxidation of 128

| Expt | Oxidant | Acetonitrile | Result of oxidation |
|---|---|---|---|
| D-169-177-1 | PCC | Wet | No oxidation |
| D-173-89C | PCC | Dry | Oxidation to 129 |
| D-173-89D | MnO₂ | Wet | No oxidation |
| D-173-89A | MnO₂ | Dry | No oxidation |
| D-173-89E | Oxone | Wet | 128a mostly consumed; gave |
| | | | product containing 29% 129, |
| | | | 15.7% 128a and 36.3% |
| | | | unknown (RRT to 129 0.80) |
| D-173-89B | Oxone | Dry | No oxidation |

D. Tracking of Residual Metals in 129

A sample of one of the lots of recrystallized 129 (lot D-169-165-3) was submitted for residual metal analysis by ICP-OES. The results were 2 ppm Cu and 81 ppm Cr. Therefore it is contemplated that according to this process, the process additional steps to remove residual metals will not be needed.

E. Development of CAD™ HPLC Method for Detecting 129

The charged aerosol detection (CAD™) HPLC was set up for detecting DCA. The retention time for 129 was consistently at 15.87 min. A dose response study for 129 showed a good linear fit for a log (area response) versus log (concentration) as would be expected for a CAD detector. Retention time for chromatographed 128a was determined to be 21.6 min (RRT 1.36); this peak appears to be a double peak-possibly due to epimers of the alcohol. Retention time for 128 was determined to be 29.4 min (RRT 1.85). Both batches of 128 gave the same retention time. Sample of 129 was run and its purity was 87.2% (AUC) with 1.75 C-20 epimer (RRT 1.19); this includes a shoulder peak not present in samples of recrystallized 129 (purity 96.3% with 3.7% c-20 epimer). HPLC of the mother liquors from 129 recrystallization (purity 33.8%) is also included for reference.

### Example 4

### Converting Compound 129 To DCA

In Scheme 1 below, there is provided a scheme for the synthesis and purification of DCA from compound 1.

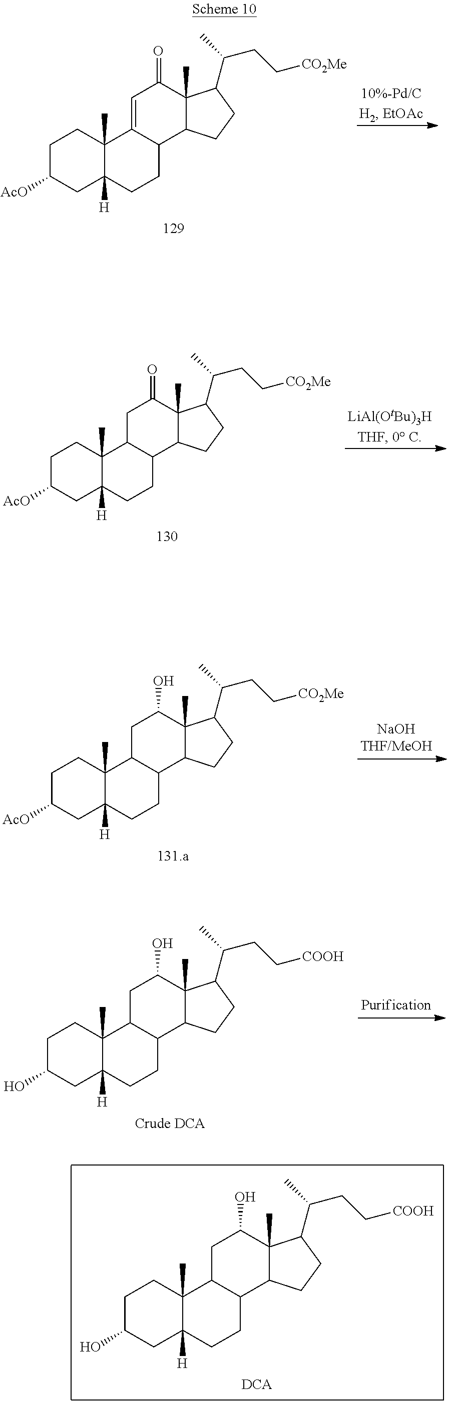

### A. Conversion of Compound 129 to Compound 130:

### Method A1

10% Pd/C (900 mg) was added to a solution of compound 129 (2.0 g, 4.5 mmol) in EtOAc (150 mL) and the resulting slurry was hydrogenated in a Parr apparatus (50 psi) at 50° C. for 16 h. At this point the reaction was determined to be complete by TLC. The mixture was filtered through a small plug of Celite® and the solvent was removed under vacuum, providing compound 130 (1.6 g, 80% yield) as a white solid.

TLC: p-anisaldehyde charring, Rf for 130=0.36.

TLC mobile phase: 20%-EtOAc in hexanes.

¹H NMR (500 MHz, CDCl₃): δ=4.67-4.71 (m, 1H), 3.66 (s, 3H), 2.45-2.50 (t, J=15 Hz, 2H), 2.22-2.40 (m, 1H), 2.01 (s, 3H), 1.69-1.96 (m, 9H), 1.55 (s, 4H), 1.25-1.50 (m, 8H), 1.07-1.19 (m, 2H), 1.01 (s, 6H), 0.84-0.85 (d, J=7.0 Hz, 3H).

¹³C NMR (125 MHz, CDCl₃): δ=214.4, 174.5, 170.4, 73.6, 58.5, 57.4, 51.3, 46.4, 43.9, 41.2, 38.0, 35.6, 35.5, 35.2, 34.8, 32.0, 31.2, 30.4, 27.4, 26.8, 26.2, 25.9, 24.2, 22.6, 21.2, 18.5, 11.6.

Mass (m/z)=447.0 [M⁺+1], 464.0 [M⁺+18].

IR (KBr)=3445, 2953, 2868, 1731, 1698, 1257, 1029 cm⁻¹.

m.p.=142.2-144.4° C. (from EtOAc/hexanes mixture).

[α]_{D}=+92 (c=1% in CHCl₃).

ELSD Purity: 96.6%: Retention time=9.93 (Inertsil ODS 3V, 250×4.6 mm, 5 um, ACN: 0.1% TFA in water (90:10)

### Method A2

A slurry of 10% Pd/C (9 g in 180 mL of ethyl acetate) was added to a solution of compound 129 (36 g, 81 mmol) in EtOAc (720 mL) and the resulting slurry was treated with hydrogen gas (50 psi) at 45-50° C. for 16 h. (A total of 1080 mL of solvent may be used). At this point the reaction was determined to be complete by HPLC(NMT 1% of compound 129). The mixture was filtered through Celite® (10 g) and washed with ethyl acetate (900 mL). The filtrate was concentrated to 50% of its volume via vacuum distillation below 50° C. To the concentrated solution was added pyridinium chlorochromate (20.8 g) at 25-35° C. and the mixture was stirred for 2 h at 25-35° C., when the reaction completed by HPLC (allylic alcohol content is NMT 1%).

The following process can be conducted if compound 129 content is more than 5%. Filter the reaction mass through Celite® (10 g) and wash with ethyl acetate (360 mL). Wash the filtrate with water (3×460 mL) and then with saturated brine (360 mL). Dry the organic phase over sodium sulphate (180 g), filter and wash with ethyl acetate (180 mL). Concentrate the volume by 50% via vacuum distillation below 50° C. Transfer the solution to a clean and dry autoclave. Add slurry of 10% palladium on carbon (9 g in 180 mL of ethyl acetate). Pressurize to 50 psi with hydrogen and stir the reaction mixture at 45-50° C. for 16 h.

Upon complete consumption of compound 129 by HPLC (the content of compound 129 being NMT 1%), the reaction mixture was filtered through Celite® (10 g) and the cake was washed with ethyl acetate (900 mL). The solvent was concentrated to dryness via vacuum distillation below 50° C. Methanol (150 mL) was added and concentrated to dryness via vacuum distillation below 50° C. Methanol (72 mL) was added to the residue and the mixture was stirred for 15-20 min at 10-15° C., filtered and the cake was washed with methanol (36 mL). The white solid was dried in a hot air drier at 45-50° C. for 8 h to LOD being NMT 1% to provide compound 230 (30 g, 83.1% yield).

### B. Conversion of Compound 130 to Compound 131.a

### Method B1

A THF solution of lithium tri-tert-butoxyaluminum hydride (1M, 22.4 mL, 22.4 mmol) was added drop wise to a solution of compound 130 (2.5 g, 5.6 mmol) in THF (25 mL) at ambient temperature. After stirring for an additional 4-5 h, the reaction was determined to be complete by TLC. The reaction was quenched by adding aqueous HCl (1M, 10 mL) and the mixture was diluted with EtOAc (30 mL). The phases were separated and the organic phase was washed sequentially with water (15 mL) and saturated brine solution (10 mL). The organic phase was then dried over anhydrous Na₂SO₄ (3 g) and filtered. The filtrate was concentrated under vacuum and the resulting solid was purified by column chromatography [29 mm (W)×500 mm (L), 60-120 mesh silica, 50 g], eluting with EtOAc/hexane (2:8) [5 mL fractions, monitored by TLC with p-anisaldehyde charring]. The fractions containing the product were combined and concentrated under vacuum to provide compound 131.a (2.3 g, 91%) as a white solid.

TLC: p-anisaldehyde charring, Rf for 131.a=0.45 and Rf for 130=0.55.

TLC mobile phase: 30%-EtOAc in hexanes.

¹H NMR (500 MHz, CDCl₃): δ=4.68-4.73 (m, 1H), 3.98 (s, 1H), 3.66 (s, 3H), 2.34-2.40 (m, 1H), 2.21-2.26 (m, 1H), 2.01 (s, 3H), 1.75-1.89 (m, 6H), 1.39-1.68 (m, 16H), 1.00-1.38 (m, 3H), 0.96-0.97 (d, J=5.5 Hz, 3H), 0.93 (s, 3H), 0.68 (s, 3H).

¹³C NMR (125 MHz, CDCl₃): δ=174.5, 170.5, 74.1, 72.9, 51.3, 48.1, 47.2, 46.4, 41.7, 35.8, 34.9, 34.7, 34.0, 33.5, 32.0, 30.9, 30.8, 28.6, 27.3, 26.8, 26.3, 25.9, 23.4, 22.9, 21.3, 17.2, 12.6

Mass (m/z)=449.0 [M⁺+1], 466.0 [M⁺+18].

IR (KBr)=3621, 2938, 2866, 1742, 1730, 1262, 1162, 1041, cm⁻¹.

m.p=104.2-107.7° C. (from EtOAc).

[α]_{D}=+56 (c=1% in CHCl₃).

ELSD Purity: 97.0%: Retention time=12.75 (Inertsil ODS 3V, 250×4.6 mm, 5 um, ACN:Water (60:40)

### Method B2

A THF solution of lithium tri-tert-butoxyaluminum hydride (1M, 107.6 mL, 107.6 mmol) was added over 1 h to a solution of compound 130 (30.0 g, 67 mmol) in dry THF (300 mL) at 0-5° C. After stirring for an additional 4 h at 5-10° C., the reaction was determined to be complete by HPLC(NMT 1% of compound 130). The reaction was cooled to 0-5° C. and quenched by adding 4N HCl (473 mL). The phases were separated. The aqueous layer was extracted with DCM (2×225 mL) and the combined organic phase was washed sequentially with water (300 mL) and saturated brine solution (300 mL). The organic phase was then was concentrated to dryness by vacuum distillation below 50° C. Methanol (150 mL) was added to the residue and concentrated to dryness by vacuum distillation below 50° C. Water (450 mL) was then added to the residue and the mixture was stirred for 15-20 min., filtered and the cake was washed with water (240 mL). The white solid was dried in a hot air drier at 35-40° C. for 6 h to provide compound 131.a (30 g, 99.6%).

### C. Conversion of Compound 131.a to Crude DCA:

To a solution of 131.a in MeOH (4 vol) and THF (4 vol) was added a solution of NaOH (4.0 equiv) in DI water (5 M) maintaining the temperature below 20° C. HPLC analysis after 20 hours at 20-25° C. indicated <0.5% AUC of 131.a and the two intermediates remained. The reaction was deemed complete, diluted with DI water (10 vol) and concentrated to ˜10 volumes. The sample was azeotroped with 2-MeTHF (2×10 vol) and assayed by ¹H NMR to indicate MeOH was no longer present. The rich aqueous phase was washed with 2-MeTHF (2×10 vol) and assayed by HPLC to indicate 0.3% AUC of the alcohol impurity remained. The aqueous phase was diluted with 2-MeTHF (10 vol) and adjusted to pH=1.7−2.0 using 2 M HCl (˜4 vol). The phases were separated and the 2-MeTHF phase was washed with DI water (2×10 vol). The 2-MeTHF phase was filtered over Celite and the filter cake was washed with 2-MeTHF (2 vol). The 2-MeTHF filtrate was distillated to ˜10 volumes and azeotroped with n-heptane containing Statsafe™ 5000 (3×10 vol) down to ˜10 vol. The mixture was assayed by ¹H NMR to indicate <5 mol % of 2-MeTHF remained relative to n-heptane. The slurry was held for a minimum of 2 hours at 20-25° C. and filtered. The filter cake was washed with n-heptane (2×10 vol) and conditioned under vacuum on the Nütsche filter with N₂ for a minimum of 1 hour to afford DCA-crude as white solids. Purity=94.6% (by HPLC). HPLC analysis for DS-DCA (NMT 5% AUC).

### D. Recrystallization of DCA

DCA-crude was diluted with 2 mol % MeOH in CH₂Cl₂ (25 vol) and heated to 35-37° C. for 1 hour. The slurry was allowed to cool to 28-30° C. and filtered. The filter cake was washed with CH₂Cl₂ (5 vol) and dried under vacuum at 40° C. to afford DCA. HPLC analysis for DS-DCA (NMT 0.15% AUC).

DCA was dissolved in 10% DI water/EtOH (12 vol), polish filtered over Celite and washed with 10% DI water/EtOH (3 vol). The resulting 15 volume filtrate was added to DI water (30 vol) and a thin white slurry was afforded. The slurry was held for 24 hours, filtered, washed with DI water (20 vol) and dried under vacuum at 40° C. to afford pure DCA. OVI analysis for CH₂Cl₂, EtOH, n-heptane, MeOH and MeTHF was conducted to ensure each solvent was below ICH guideline. KF analysis conducted (NMT 2.0%). Purity=99.75% (by HPLC). Yield from DCA-crude=59%.

### Example 5

### Purification of DCA Containing Low Levels of DS-DCA

Crystallization of DCA was tested in EtOH/H₂O to evaluate the recovery and the extent of purification of DCA. About 0.50 g of DCA (0.54% area under the curve (AUC) of DS-DCA) was added to 14 vials. As tabulated below, different volumes of EtOH and deionized water (Water #1, 10% v/v of the EtOH amount to avoid potential ester formation) were added to dissolve the material with stirring at 70° C., giving a clear solution. Additional deionized water (Water #2) was added until turbidity was observed. The mixture was heated at 70° C. and then polish-filtered using syringe filters (13 mm, 0.45 μm, PVDF Durapore) into preheated vials at 70° C. The contents were cooled to 60° C. and about 5 mg (1 wt %) of Form C seeds was added to each vial. The crystallization conditions and results are tabulated below.

| | DCA | EtOH | Water #1 | Water #2 | Water #3 | Yield | HPLC | HPLC |
|---|---|---|---|---|---|---|---|---|
| Code | (mg) | (mL) | (mL) | (mL) | (mL) | (mg) | DCA | DS-DCA |
| TTO-A-39-1 | 502 | 4.5 | 0.45 | 3.0 | 0.0 | 357 | 99.76 | 0.03 |
| TTO-A-39-2 | 501 | 4.5 | 0.45 | 3.0 | 0.5 | 400 | 99.82 | ND |
| TTO-A-39-3 | 502 | 4.5 | 0.45 | 3.0 | 1.0 | 416 | 99.60 | ND |
| TTO-A-39-4 | 501 | 4.5 | 0.45 | 3.0 | 1.5 | 421 | 99.64 | 0.05 |
| TTO-A-39-5 | 501 | 4.5 | 0.45 | 3.0 | 2.0 | 424 | 99.54 | ND |
| TTO-A-35-1 | 504 | 4.5 | 0.45 | 3.0 | 3.0 | 430 | 99.17 | 0.13 |
| TTO-A-35-2 | 501 | 4.5 | 0.45 | 3.1 | 3.9 | 427 | 98.74 | 0.14 |
| TTO-A-35-3 | 501 | 4.5 | 0.45 | 3.0 | 5.0 | 432 | 98.69 | 0.18 |
| TTO-A-35-4 | 505 | 4.5 | 0.45 | 3.1 | 5.9 | 433 | 98.65 | 0.22 |
| TTO-A-35-5 | 502 | 4.5 | 0.45 | 3.0 | 7.0 | 410 | 98.38 | 0.33 |
| TTO-A-35-6 | 502 | 3.8 | 0.38 | 1.8 | 4.0 | 434 | 98.58 | 0.22 |
| TTO-A-35-7 | 501 | 4.45 | 0.445 | 3.3 | 3.6 | 432 | 98.76 | 0.17 |
| TTO-A-35-8 | 504 | 5.3 | 0.53 | 3.6 | 5.8 | 435 | 98.92 | 0.18 |
| TTO-A-35-9 | 503 | 3.7 | 0.37 | 1.5 | 5.1 | 427 | 98.67 | 0.21 |

The seeds remained undissolved in experiments TTO-A-35-1 to TTO-A-35-5 but dissolved in TTO-A-35-6 to TTO-A-35-9 and TTO-A-39-1 to TTO-A-39-5. The contents were cooled to 55° C. About 5 mg (1 wt %) of seeds was added to TTO-A-35-6 to TTO-A-35-9 and TTO-A-39-1 to TTO-A-39-5. The seeds remained in lots TTO-A-35-6 to TTO-A-35-8 but dissolved in lot TTO-A-35-9 and TTO-A-39-1 to TTO-A-39-5. The contents were cooled to 50° C. About 5 mg (1 wt %) of seeds was added to TTO-A-35-9 and TTO-A-39-1 to TTO-A-39-5. The seeds remained in TTO-A-39-1 to TTO-A-39-5 but dissolved in lot TTO-A-35-9. The contents were cooled to 45° C. and about 5 mg (1 wt %) of seeds (lot 02110037) was added to TTO-A-35-9. The seeds remained undissolved.

All the experiments were cooled at 10° C./h to 20° C. and left to stir overnight. A final portion of deionized water (Water #3) was added and the mixtures were stirred for 3 hours. The solids were filtered and XRPD analysis showed all were Form C. After drying under vacuum at 65° C. for 60 hours, XRPD showed all the solids converted to Form B. HPLC analysis results are tabulated above and described as follows.

When EtOH was 4.5 mL and the amount of Water #3 was less than 3.9 mL, DS-DCA level was reduced from 0.54% AUC to <0.15% AUC. When Water #3 was at 1.0-3.9 mL, the recovery was at a maximum level and the recovery remained unchanged even when a higher amount of water anti-solvent was added. When Water #3 was less than 1.0 mL, the recovery was lower. These results indicated that the experiment TTO-A-39-5 was the most robust conditions on DS-DCA removal and recovery when DCA lot 31DJG054A (containing 0.54% AUC DS-DCA) was used. In the Experiments TTO-A-35-6 to TTO-A-35-9, extra water was added and the results were consistent with the observation that high water ratio deteriorates purification.

The experiment TTO-A-39-5 was repeated on a 5 g DCA scale with minor changes on polish filtration protocol (TTO-A-43) and on a 1 g scale without performing polish-filtration and seeding (TTO-A-44). HPLC analysis showed successful purification for the 5 g experiment as well as the 1 g experiment, as described below, indicating that seeding and polish filtration steps are not critical steps for purification and can be skipped to further simplify the process.

TTO-A-43: DCA (5.0 g, 0.54% AUC of DS-DCA) was added to a 40-mL vial and dissolved in 10% water in EtOH (35 mL, 7 vol) at 70° C. The solution was filtered through a syringe filter (13 mm, 0.45 μm, PVDF Durapore) into a 250 mL round bottom flask equipped with stir bar. The solution was heated to 70° C. The vial was rinsed with 15 mL of 10% water in EtOH and filtered into the flask. DI Water (30 mL) was added slowly maintaining temperature above 60° C. (approximately 15 minutes for completing the addition). The solution was cooled to 60° C. and Form C seed crystal (50 mg or 1 wt %, lot 02110037) was added as a slurry in 1.5 mL of DI-water. A slightly turbid solution was observed. The batch was cooled to ambient temperature at 10° C./h and allowed to stir over night. DI water (20 mL) was then added slowly via an addition funnel over a period of 30 minutes. The resulting solution was stirred at ambient temperature for 3 hours and filtered. The solid was analyzed by XRPD and dried in vacuum at 62° C., giving DCA in 92.4% yield (4.62 g). XRPD pattern indicated polymorph conversion from Form C to Form B. HPLC analysis showed 99.75% AUC purity containing only 0.06% AUC of DS-DCA.

TTO-A-44: DCA (1.0 g, 0.54% AUC of DS-DCA) was added to a 40 mL vial. EtOH (9.0 mL) and DI water (0.9 mL) were added to dissolve the solids with stirring and heating to 70° C. to achieve a clear solution. DI water (6.0 mL) was added and turbidity was observed. It was cooled to 20° C. at 10° C./h and left to stir overnight. DI water (4.0 mL) was added over 30 minutes. The contents were left to stir for 3 hours and filtered. The solid was analyzed by XRPD and dried in vacuum at 62° C., giving DCA in 83.2% yield (0.83 g). XRPD pattern indicated polymorph conversion from Form C to Form B. HPLC analysis showed 99.80% AUC purity containing only 0.06% AUC of DS-DCA.

1-9. (canceled)

10. A process of oxidizing a 12-position methylene group of a steroid which methylene group is adjacent to a Δ-9,11-ene, the method comprising contacting the steroid containing the methylene group with tertiarybutyl hydroperoxide and CuI under conditions to provide a 12-hydroxy Δ-9,11-ene steroid and optionally a 12-keto Δ-9,11-ene steroid.

11. The process of claim 10, further comprising contacting the 12-hydroxy Δ-9,11-ene steroid with pyridinium chlorochromate under conditions to provide the 12-keto Δ-9,11-ene steroid.

12. (canceled)

13. A crystalline anhydrate polymorph of DCA.

14. The anhydrate polymorph of DCA of claim 13, which is in Form B.

15. A crystalline Form B polymorph of DCA characterized by 1, 2, or 3 PXRD peaks selected from the group consisting of 6.7, 7.3, 7.4, 8.4, 9.3, 11.2, 12.9, 13.9, 14.4, 14.6, 14.8, 15.8, 16.0, 16.9, and 17.8° 2theta.

16. The Form B polymorph of claim 15, characterized by a PXRD pattern substantially as shown in FIG. 2.

17. The polymorph of claim 13 admixed with a pharmaceutically acceptable excipient.

18. (canceled)
